(19) United States
(12) Patent Application Publication
(10) Pub. No.: US 2019/0341151 A1
(43) Pub. Date: Nov. 7, 2019

(54) HEALTH SUPPORT SYSTEM

(71) Applicant: RENESAS ELECTRONICS CORPORATION, Tokyo (JP)

(72) Inventors: Shoichi HAMADA, Tokyo (JP); Kakeru KIMURA, Tokyo (JP); Hirohisa IMAMURA, Tokyo (JP); Koji HIRANO, Tokyo (JP)

(21) Appl. No.: 16/380,694

(22) Filed: Apr. 10, 2019

(30) Foreign Application Priority Data
May 7, 2018 (JP) 2018-089390

(51) Int. Cl. G16H 50/30; G16H 10/60

(52) U.S. Cl. CPC G16H 50/30; G16H 10/60

(57) ABSTRACT

A health support system that facilitates estimation of a user's health condition status and improvement of the health condition status is constructed. The health support system includes a first sensing device for acquiring biometric information of a user, a first actuator device operating based on abstracted data, and a first server connected to the first sensing device and the first actuator device via a network in a daily living space in which the user lives a daily life. The first sensing device or the server generates the abstracted data based on the biometric information. The abstracted data is classified by estimating a health condition of the user. The first actuator device facilitates improving a health condition of the user.

### CROSS-REFERENCE TO RELATED APPLICATIONS

The disclosure of Japanese Patent Application No. 2018-089390 filed on May 7, 2018 including the specification, drawings and abstract is incorporated herein by reference in its entirety.

### BACKGROUND

The present disclosure relates to a health support system and can be applied to a health support system using, for example, a home electric appliance.

Home appliances have widely penetrated daily households and have greatly changed people's lives. For example, white appliances such as refrigerators and washing machines have greatly improved household efficiency, and video and audio equipment such as televisions and stereos have provided new entertainment. It has been proposed that a home electric appliance system is configured by a plurality of home electric appliances and a home server (for example, Japanese Patent Laid-Open No. 2002-315079) or a home electric appliance system is configured by connecting a plurality of home electric appliances via a network (for example, Japanese Patent Laid-Open No. 2015-184563).

### SUMMARY

A health support system that facilitates estimation of the health condition of the user and improvement of the health condition will be constructed by using a device having a function of supporting activities in daily life (for example, home electric appliances, housing facilities, in-vehicle devices). Other objects and novel features will become apparent from the description of the present disclosure and the accompanying drawings.

The typical aspects of the present disclosure will be briefly described below. That is, the health support system estimates and abstracts the health condition of the user based on the sensing data sensed by the sensing device in the daily living space, and the actuator device facilitates the improvement of the health condition of the user based on the abstracted health condition data.

According to the health support system, it is possible to facilitate estimation of the health condition of the user and improvement of the health condition.

### BRIEF DESCRIPTION OF THE DRAWINGS

FIG. 1 is a diagram illustrating an example of the configuration of a health support system in a daily living space.

FIG. 2 is a block diagram illustrating a schematic configuration of a sensing device, an actuator device, and a bi-function device of FIG. 1.

FIG. 3 is a block diagram of a microcomputer (MCU) for processing a sensor embedded in a sensing device and its sensing information of FIG. 2.

FIG. 4 is a block diagram of an MCU and an actuator embedded in an actuator device of FIG. 2.

FIG. 5A is a diagram depicting the structure of the home and the out-of-the-home where a health condition information database is brought out or back from.

FIG. 5B is a diagram illustrating a configuration example of an actuator device, a bi-function device, and a sensing device of the hotel of FIG. 5A.

FIG. 5C is a diagram showing an example of the actuator device, the bi-function device, and the sensing device of the accommodation in FIG. 5A.

FIG. 6 is a diagram illustrating a health support system at home and at a hotel.

FIG. 7A is a diagram showing the content of records in the health condition information database between the home and the hotel.

FIG. 7B is an enlarged view showing the recorded content of the health condition information database of the home server of FIG. 7A.

FIG. 7C is an enlarged view showing the recorded content of the health condition information database of a terminal of FIG. 7A.

FIG. 7D is an enlarged view showing the recorded content of the health condition information database of the home server of FIG. 7A.

FIG. 8 shows a diagram of a hotel in a cold area where the database from a home in a warm area is brought out.

FIG. 9A is a diagram showing the operation of a home server in a home, a terminal in a hotel, and a home server in a residential accommodation in a sequence diagram.

FIG. 9B is a sequence diagram illustrating the operation of a terminal, a sensing device, and an actuator device of a hotel.

FIG. 10 is a flowchart of the device control processing according to the stomach condition.

FIG. 11 is a flowchart showing the data acquisition processing using the toilet.

FIG. 12 is a diagram representing a possible combination of sensing data from the toilet 140a.

FIG. 13A is a flowchart of abstraction processing of the stomach condition

FIG. 13B is another flowchart of abstraction processing of the stomach condition.

FIG. 14 shows a table for converting sensing data of the toilet 140a to intermediate data.

FIG. 15 shows a table for converting sensing data of the toilet 240a to intermediate data.

FIG. 16 is a diagram showing a table for converting sensing data of toilet 340a into intermediate data.

FIG. 17 shows the table where the interim data is accumulated in units of one day.

FIG. 18 is a table showing the conditions for abstracting the physical condition of the stomach.

FIG. 19 is a diagram illustrating an example of a resident's physical condition record when diarrhea is detected in a certain period.

FIG. 20 is a diagram showing the number of times of diarrhea.

FIG. 21 is a flowchart showing the operation of an actuator device, etc. in a home.

FIG. 22 is a flowchart showing an outline of a cold judgement.

FIG. 23 is a diagram illustrating a history of changes in body temperature of a user.

FIG. 24 is a diagram showing the judgement of a febrile cold.

FIG. 25 is a flowchart of the heat-generating cold detection processing.

FIG. 26A is a diagram illustrating a history of the user's nasal opening voice spectrum.

FIG. 26B is a diagram showing the history of the closed nasal voice spectrum.

FIG. 27 is a diagram showing an example of a nasal closing voice cold judgement table.

FIG. 28 is a flowchart of the nasal closing voice cold detection processing.

FIG. 29 is a diagram showing the sound waveform pattern of a user's cough.

FIG. 30 is a diagram showing an example of a cough frequency cold judgement table.

FIG. 31 is a flowchart of the cough frequency cold detection processing.

FIG. 32 is a diagram showing the sound waveform pattern of a user's sneeze.

FIG. 33 is a diagram showing an example of a nasal cold sound frequency judgement table.

FIG. 34 is a flowchart of the nasal cold sound frequency detection processing.

FIG. 35 is a diagram illustrating a sneezing action pattern of a user.

FIG. 36 is a diagram showing an example of a judgement table of the number of nasal colds.

FIG. 37 is a flowchart for detecting the number of nasal cold action.

FIG. 38 is an example of comprehensive judgement of abstracted data from the cold level derived from each sensing result.

FIG. 39 is a flowchart showing the processing of stress level abstraction.

FIG. 40 shows the table on which intermediate data for abstraction from sensing data are extracted.

FIG. 41 is a diagram showing an example of a stress judgement table.

FIG. 42 is a diagram illustrating an example of bringing out a health condition information database when leaving the home using a taxi.

FIG. 43 is a diagram illustrating a configuration of an air cushion that is an example of an actuator device.

### DETAILED DESCRIPTION

Embodiments and examples will be described below with reference to the drawings. However, in the following description, the same components are denoted by the same reference numerals, and a repetitive description thereof may be omitted.

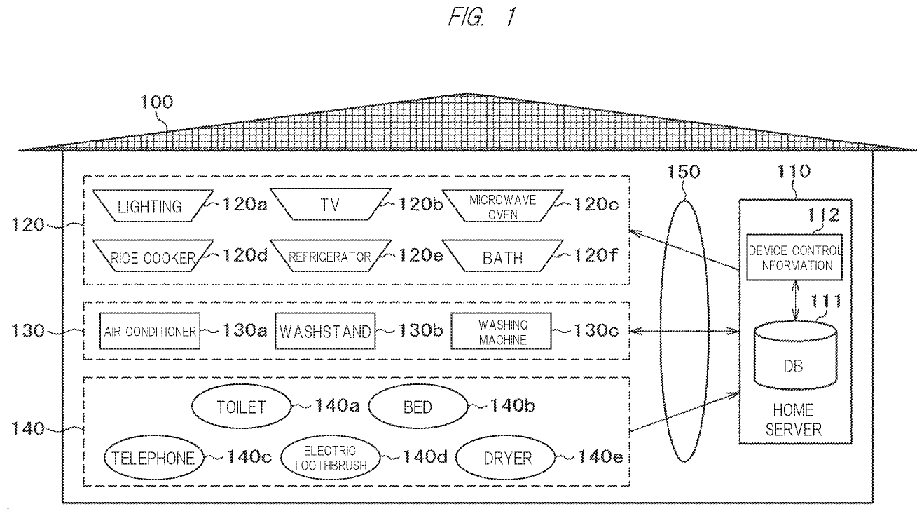

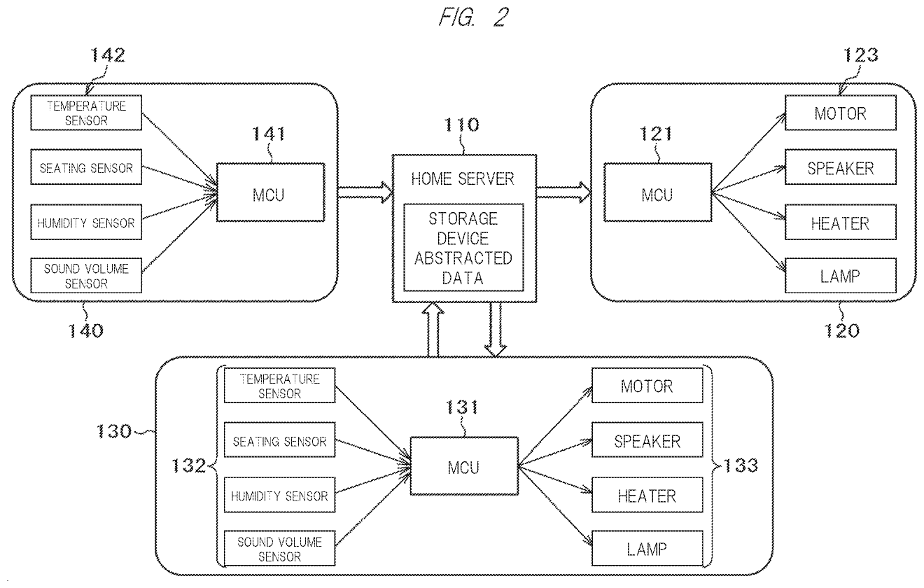

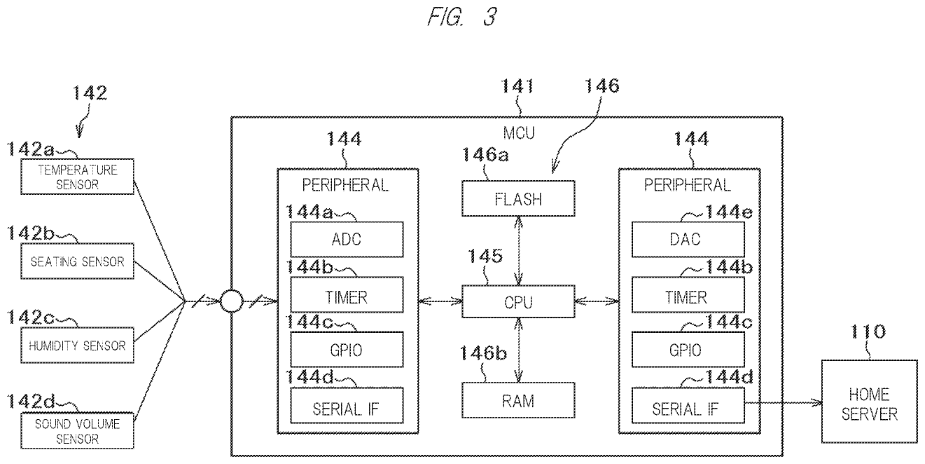

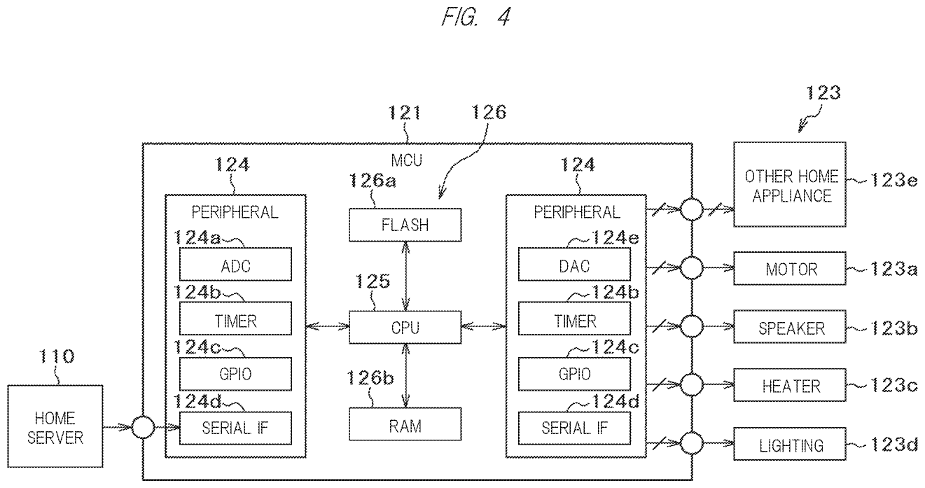

A health support system in a living space where a daily life is performed (hereinafter, referred to as a daily life space) will be described with reference to FIGS. 1 to 4. FIG. 1 is a diagram showing a configuration example of a health support system in a daily living space. FIG. 2 is a block diagram showing a schematic configuration of the sensing device, the actuator device, and the bi-function device of FIG. 1. FIG. 3 is a block diagram of a sensor incorporated in the sensing device of FIG. 2 and a microcomputer for processing sensing information. FIG. 4 is a block diagram of an MCU and an actuator device incorporated in the actuator device of FIG. 2.

As shown in FIG. 1, a home 100, which is an example of a daily living space, includes a home server 110, which is an example of a server, an actuator device 120, a bi-function device 130, a sensing device 140, and a home network 150, which is an example of a network connecting these devices.

The home server 110 includes a CPU, an input unit, a display unit, a communication unit, and a storage unit, and the storage unit stores a health condition information database 111 and device control information 112 which is optimum control information of the device analyzed from the health condition information database 111. The health condition information database 111 of the home server 110 is master data.

The communication unit of the home server 110 has a communication interface (IF) with the actuator device 120, the bi-function device 130, and the sensing device 140 installed in the home 100 and has a function of transmitting and receiving data. The actuator device 120, the bi-function device 130, and the sensing device 140 all have a communication IF and communicate with the home server 110 via the home network 150 according to the function classification.

The actuator device 120 is a device that performs a passive operation, for example, a function of displaying information or a device operation by performing control from the outside and receives a control signal of the device control information 112 from the home server 110 and changes settings and operations. For example, household appliances such as the lighting 120a, TV (television) 120b, microwave oven 120c, rice cooker 120d, and refrigerator 120e, and housing facilities such as the bath 120f correspond to the actuator device 120, and the optimal operation is proposed according to the health condition of the resident who is the user of the device received from the home server 110.

The sensing device 140 is a device having a function of acquiring sensing information by a sensor or the like built in each device and transmitting data of an operation condition to the outside and transmits the sensing information to the home server 110. For example, home facilities such as a toilet 140a, furniture such as a bed 140b, and home appliances such as a telephone 140c, an electric toothbrush 140d, and a dryer 140e correspond to the sensing device 140, acquire biometric information such as body temperature, pulse rate, heartbeat, tone of voice, coughing frequency, and the like, estimate abstracted health condition information (hereinafter referred to as abstracted data), and transmit the information to the home server 110. The estimation of the abstracted data may be performed by the home server 110.

The bi-function device 130 has functions of both the actuator device 120 and the sensing device 140 and is an active device capable of transmitting external control and sensor sensing information to other devices and using the sensing information for its own control. The bi-function device 130 is an actuator device having a sensing function and is a sensing device having an actuator function. The bi-function device 130 receives a control signal from the home server 110 and transmits sensing information. For example, household appliances such as the air conditioner 130a and the washing machine 130c and residential facilities such as the washstand 130b correspond to the bi-function device 130.

As shown in FIG. 2, the sensing device 140 operates the actuator device 120 and the bi-function device 130 via the home server 110. The sensing device 140 includes a microcontroller 141 and a sensor 142. The sensor 142 is, for example, a temperature sensor, a seating sensor, a humidity sensor, a sound volume sensor, or the like. The MCU 141 processes and abstracts the sensing information of the sensor 142 and transmits the processed information to the home server 110.

The actuator device 120 comprises an MCU 121 and an actuator 123. The actuator 123 is, for example, a motor, a speaker, a heater, a lamp, or the like. The MCU 121 operates the actuator 123 based on the device control data 112 from the home server 110.

The bi-function device 130 includes an MCU 131, a sensor 132, and an actuator 133. The sensor 132 is, for example, a temperature sensor, a seating sensor, a humidity sensor, a sound volume sensor, or the like. The actuator 133 is, for example, a motor, a speaker, a heater, a lamp, or the like. The MCU 131 processes and abstracts the sensing information of the sensor 132 and transmits the processed information to the home server 110. The MCU 131 operates the actuator device 123 based on the device control information 112 from the home server 110 or the information obtained by processing the sensing information and abstracting the sensing information.

As shown in FIG. 3, signals outputted from the sensors 142 (temperature sensor 142a, seating sensor 142b, humidity sensor 142c, and volume sensor 142d) are reflected into MCU 141 by a peripheral device (Peripheral) 144 (analog-to-digital converter (ADC) 144a, timer (TIMER) 144b, general-purpose I/O port (GPIO) 144c, serial interface (Serial IF) 144d, and the like) incorporated in the MCU 141. The CPU 145 analyzes and then select abstract data from the patterns of sensing information recorded in the nonvolatile memory (FLASH) 146a and the volatile memory (RAM) 145b of the storage device 146. The abstracted data is transmitted to the home server 110 at the communication modules (serial interfaces (Serial IF) 144d) of the peripheral device 144. The software programs executed by the CPU 145 are stored in the storage device 146. The abstracted data may be selected not by reading the existing data in the storage device 146, but by an Artificial Intelligence (AI) function of determining the health condition status of the user from the data obtained by accumulating and learning the sensing information in the MCU 141 and generating the abstracted data.

As shown in FIG. 4, the CPU 125 analyzes the abstracted data received from the home server 110 and selects an optimal control method stored in the nonvolatile memory (FLASH) 126a and the volatile memory (RAM) 126b of the storage device 126. The CPU 125 controls a digital-to-analog converter (DAC) 124e and a timer 124b, which are peripheral devices 124, and controls driving of a motor (Motor) 123a, a speaker (Speaker) 123b, heaters (Heater) 123c, lights (Lighting) 123d, and other home appliances (Other Home Appliance) 123e of the actuator 123. Rather than reading the pre-existing control data stored in the storage device 126, the method of selecting the control method may be an AI function for determining the optimal control method from the data accumulated and learned in the MCU 121.

Next, the operation of the health support system will be described.

The sensing device 40 and the bi-function device 130, which are installed in the home 100 and connected to the home network 150, collect the device usage status and the like by themselves. Examples of the information collected by those devices include power ON or OFF, course setting of the device, and contact information by a sensor or the like. Each device analyzes the collected data and stores the abstract physical condition of the resident in the health condition information database 111 of the home server 110 through the home network 150.

The home server 110 may accumulate and analyze the device control information 112 and store the abstract physical condition of the resident in the health condition information database 111. For example, if the same usage is used for a long period of time, such as the room temperature or the cooking setting of the rice cooker, the health condition should be recorded as good. Detecting usage and setting different from normal condition for a certain period determines that there is a problem in physical condition, and records symptoms such as stress condition or cold, and the degree of symptoms as abstract health condition information (abstracted data).

In the home 100, device control information 112 is generated based on the health condition information database 111, and control signals are transmitted to the installed actuator device 120 and the bi-function device 130 to perform automatic control or display information. It is also possible to change the setting of each device in accordance with the physical condition of the resident, or to suggest to the resident how to use the device for adjusting the physical condition.

Here, a method of generating the device control information 112 by the home server 110 and controlling the device has been described, but a configuration may be adopted in which health condition information such as physical condition stored in the health condition information database 111 is directly transmitted to the actuator device 120 and the bi-function device 130, and each device performs automatic control or provides information in accordance with the physical condition of the resident.

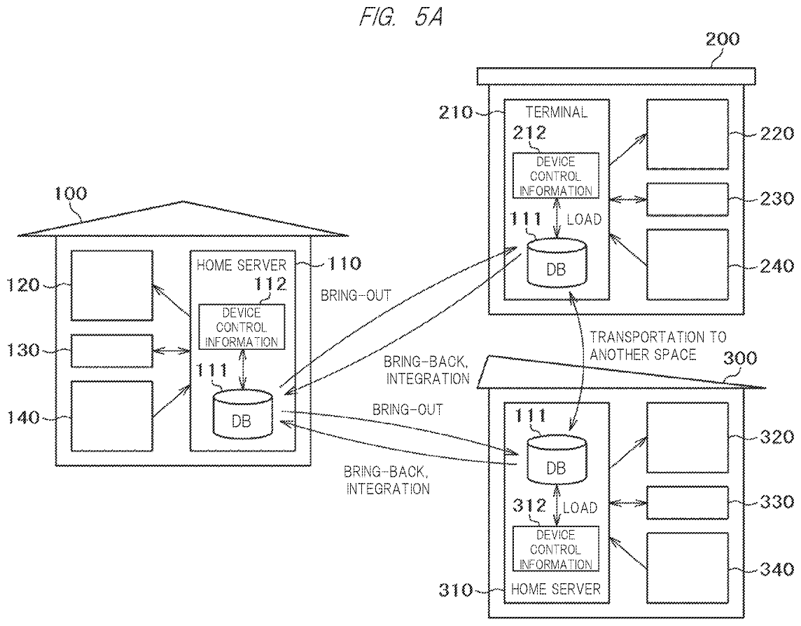

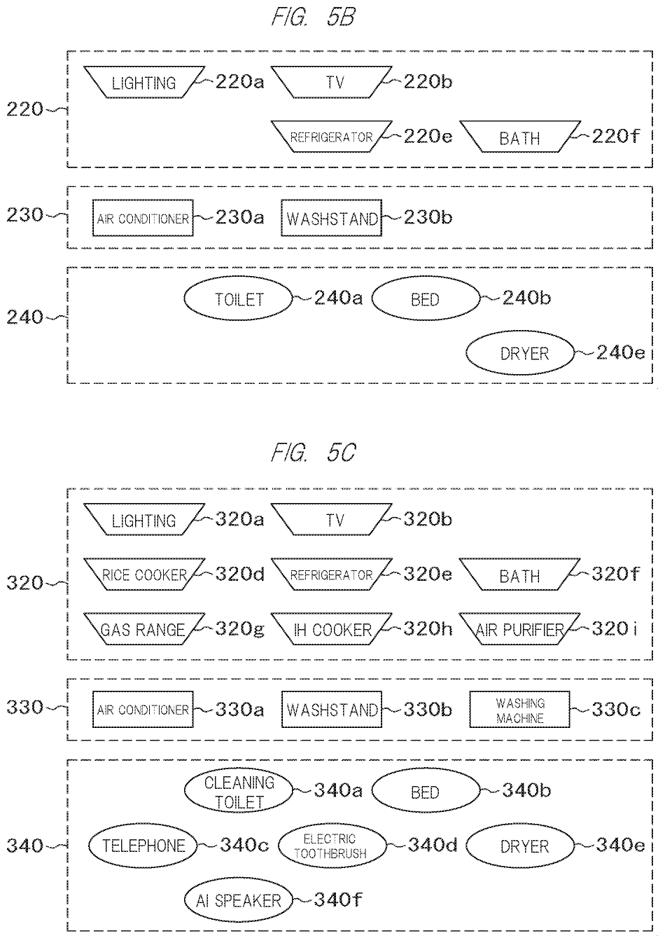

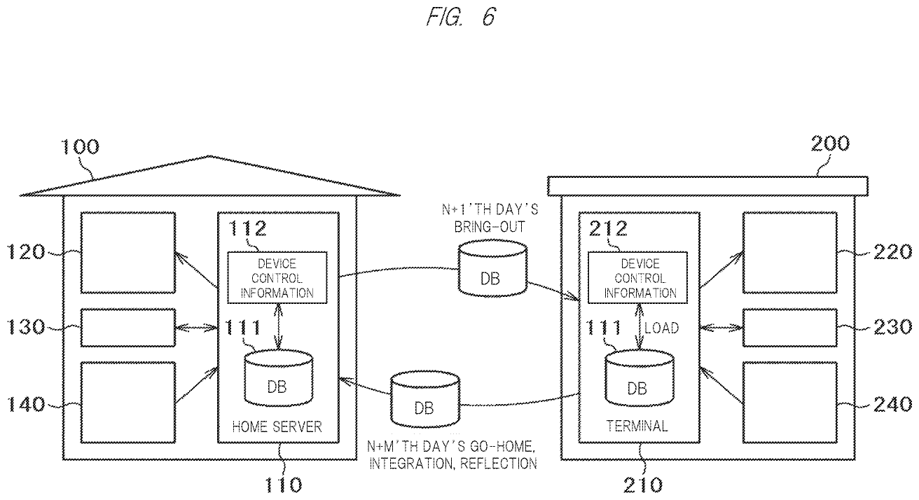

Next, a health support system in a daily living space and a non-daily living space (hereinafter, referred to as a “non-daily living space”) will be described with reference to FIGS. 5A to 5C, 6, and 7A to 7D.

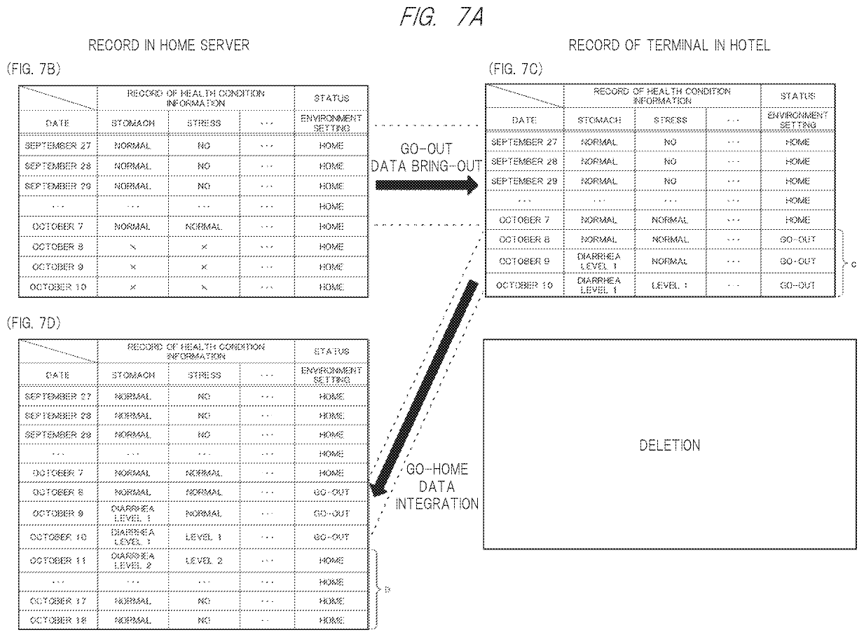

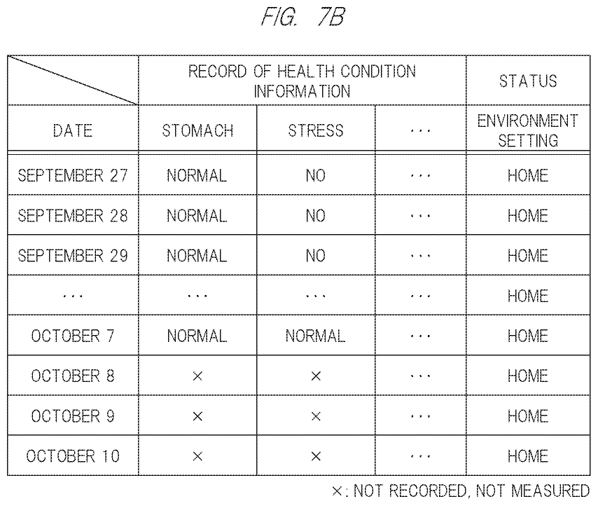

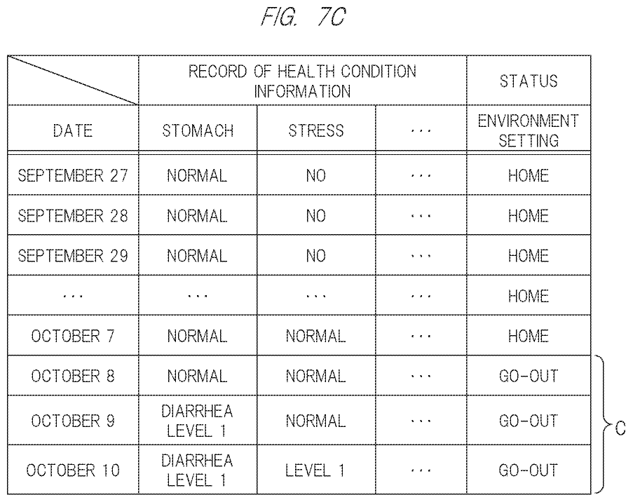

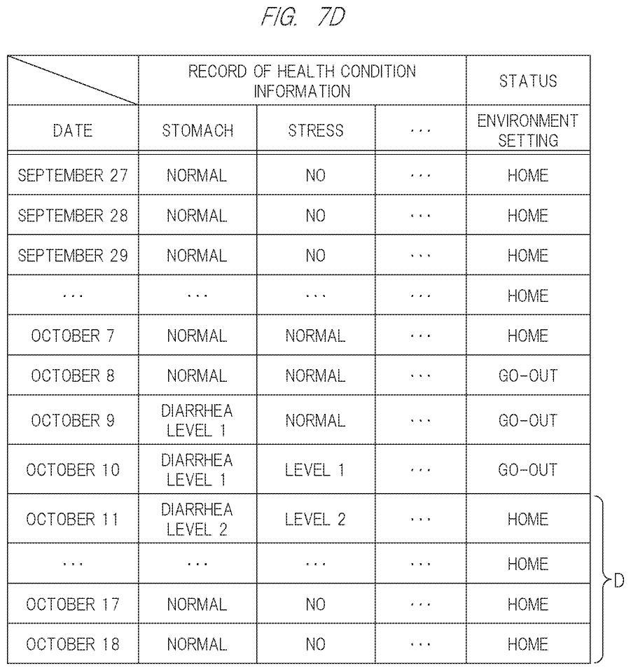

FIG. 5A is a diagram showing a configuration of a home and an outing destination from which a health condition information database is brought out. FIG. 5B is a diagram showing a configuration example of an actuator device, a bi-function device, and a sensing device of a hotel. FIG. 5C is a diagram showing an example of the actuator device, the bi-function device, and the sensing device of the accommodation. FIG. 6 is a diagram illustrating a health support system in a home and a hotel. FIG. 7A is a diagram showing the recording contents of the health condition information database between the home and the hotel. FIGS. 7B and 7D are diagrams showing the record content of the health condition information database of the home server of FIG. 7A in an enlarged manner, and FIG. 7C is a diagram showing the record content of the health condition information database of the terminal of the hotel of FIG. 7A in an enlarged manner.

A hotel 200 such as a business hotel, which is an example of a non-daily living space, is a living space having fewer devices than the home 100. The residential accommodation 300, which is another example of the non-daily living space, is a living space having a larger number of devices than the home 100. The term “residential stay” refers to staying at a part of a private residence provided for accommodation, a vacant villa, or a vacant room in an apartment building.

The terminal 210 of the hotel 200 and the home server 310 of the accommodation 300 have the same function as the home server 110 but are different in that the health condition information database 111 is deleted when it is brought home or moved to a different living space.

As shown in FIGS. 5B and 5C, the actuator device 220, the bi-function device 230, the sensing device 240, the actuator device 320, the bi-function device 330, and the sensing device 340 are of the same device type as the actuator device 120, the bi-function device 130, and the sensing device 140 respectively, but are different in the number, model, and function of the installed devices. For example, the actuator device 220 of the hotel 200 does not include a microwave oven and a rice cooker. The bi-function device 230 does not include a washing machine. The sensing device 240 does not include a telephone or an electric toothbrush. On the other hand, the actuator device 320 of the accommodation 300 does not include a microwave oven, but includes a gas range 320g, an IH cooker 320h, and an air purifier 320i. The sensing device 340 includes a cleaning toilet 340a instead of the toilet 140a, and an AI speaker 340f.

Next, the operation of the health support system at home and hotel will be described.

As shown in FIG. 7B, daily physical condition of the resident is recorded in the health condition information database 111 in the home server 110. In the “health condition information record” which is a record of physical condition, “stomach”, “stress” and the like are recorded, for example. In the health condition information database 111, “environment settings” such as home, outing, etc. are also recorded as “status”.

Control of actuator devices is performed based on abstracted data of the health condition information.

The health condition information database 111, which brings abstracted data into hotels and residential residences, can be brought from home 110 to hotel 200 through a data storage means such as non-volatile memory or a network such as the Internet.

As shown in FIG. 5A, the user brings the health condition information database 111 from the home 100 to the hotel 200 and the residential accommodation 300.

The brought-out health condition information database 111 is brought into the hotel 200 or the residential accommodation 300, which is a non-daily living space, so that it is installed in the space to perform optimum control according to the device, and it is possible to create a comfortable space for the resident.

On October 8, when the user goes out to the hotel 200 for business trip and loads the health condition information database 111 brought out of the home 100 into the terminal 210, the actuator device 220, the bi-function device 230, and the sensing device 240 are controlled by the data up to and before the day. In addition, health condition information abstracted by the bi-function device 230 and the sensing device 240 in the room of the hotel 200 is acquired from that day on, and data is additionally recorded in the brought-out health condition information database 111, as shown in C of FIG. 7C. In this case, “outing” is recorded in the “environment setting” field. When the user's physical condition is disrupted (diarrhea occurs) from October 9, the control of the actuator device 220 and the bi-function device 230 is performed in the room of the hotel 200 in accordance with the physical condition.

Even if the environment is not the same as that of the device of the home 100, the device installed in the hotel 200 and the private accommodation 300 provides optimum operation as much as possible.

In some cases, the home 100, which is a daily living space, and the hotel 200, which is a non-daily living space, and the residential accommodation 300 are installed in different types of devices. In order to absorb the performance difference, the terminal 210 of the hotel 200 and the home server 310 of the accommodation 300 automatically determine the optimal control method of each device based on the information of the devices installed in the space and the content of the loaded abstracted health condition information database 111.

The abstracted data accumulated in the hotel 200 and the residential accommodation 300 can be brought back to the home 100 for the user to continuously receive the support of the device according to the health condition.

Although the user returns home on October 11, his/her physical condition is not restored. The different setting was applied from the normal control of the device, as shown in FIG. 7C, and the abstracted data acquired in the room of the hotel 200 was recorded in the health condition information database 111. In that case, when he/she returns home and loads it into the home server 110, the data during the time of outing is integrated as shown in FIG. 7D, and then based on this information, he/she can continuously receive the same comfort environment as the environment in the room of the hotel 200 at the time of bad physical condition. D in FIG. 7D is the data recorded after returning home.

Even when the living space is changed, such as any places where the user goes out, it is possible to automatically control the device that is optimal for the current health condition based on the brought-out data, and a comfortable space can be obtained. In addition, since the device is controlled so as to reproduce a living environment suitable for the user from the health condition information and the installed device, even when the device is moved in the living space, the operation of the device is not conscious. In addition, by carrying the health condition information of the user, the data at the place of going out can be added to the data accumulated in the daily living space, so that the continuity of the data can be ensured. In addition, since the use condition of the user can be acquired at the place where the user goes out, the sign and occurrence of the physical condition change can be known at an early stage regardless of the place where the user is located, and the health can be maintained. Furthermore, since the data collected at the place of going out after returning home can be integrated with the data at home, stable health support can be obtained.

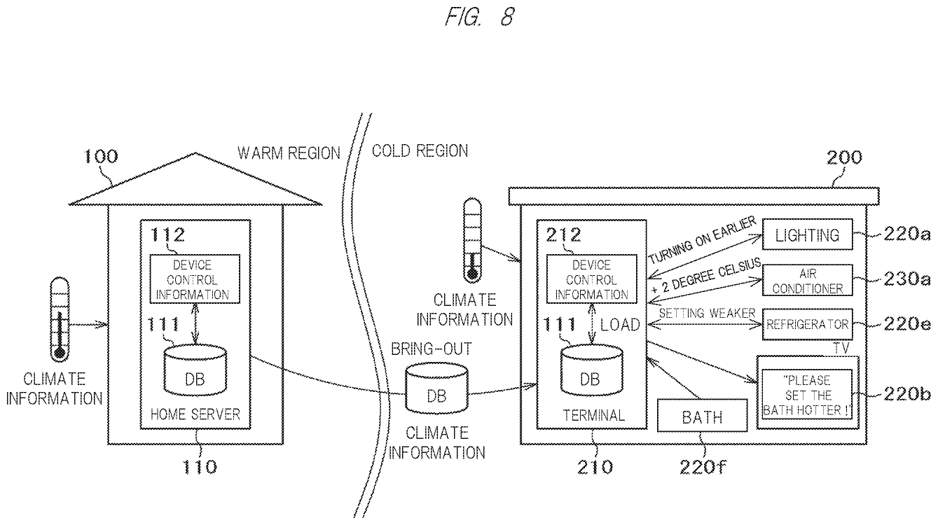

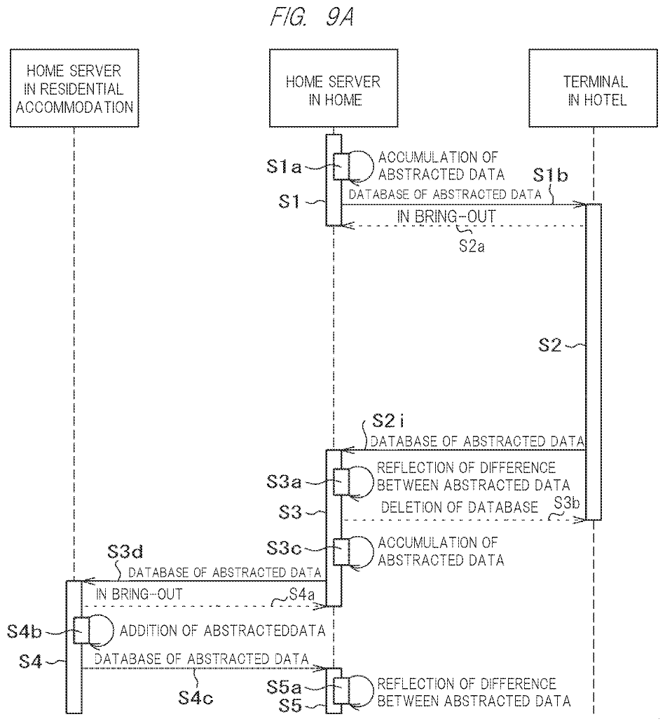

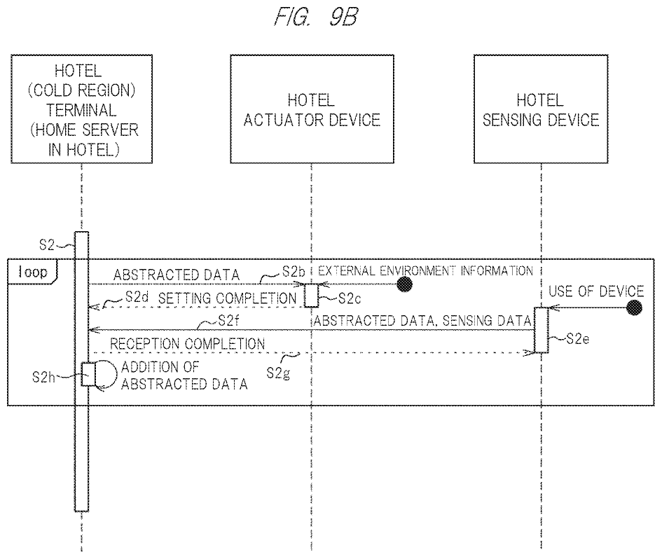

Next, an operation example of the device in the case where the health condition information database is brought out to a region having a different external environment will be described with reference to FIGS. 8, 9A, and 9B. FIG. 8 is a diagram showing a case in which a person leaves to a hotel in a cold region from a home in a warm region. FIG. 9A is a sequence diagram showing the operations of the home server of the home, the terminal of the hotel, and the home server of the accommodation. FIG. 9B is a diagram showing the operation of the sensing device, the home server, and the actuator device in a sequence diagram.

When the outside environment of the outdoor area (hotel 200) is different from the outside environment of the home 100, the setting of the device is finely adjusted in accordance with the climate condition of the outdoor area. Climate information (e.g., temperature data) of the outside environment of the home 100 is acquired and recorded in the health condition information database 111. For example, when there is a large temperature difference in the case of traveling from the home 100 to a cold region in a warm region, the hotel 200 in the cold region also acquires climate information of the outside environment, and displays that the temperature of the air conditioner should be raised, that the air conditioner should be turned on early if the sunshine time is different, that the temperature of the refrigerator should be set high, and that the bath should be warmed up.

Step S1: The home server 110 of the home 100 in the warm region acquires abstracted data from the sensing device 140 or the bi-function device 130 and accumulates it in the health condition information database (abstracted database) 111 (step S1a). The abstracted database is brought into the terminal 210 of the hotel 200 in the cold region (the home server 110 transmits the abstracted database to the terminal 210) (step S1b).

Step S2: The terminal 210 of the hotel 200 notifies the home server 110 that the database 111 is being brought out in step S2 a. In step S2b, the terminal 210 transmits the abstracted data of the health condition information database 111 to the actuator device 220.

The actuator device 220 is set with the abstracted data and the climate information as the external environment information (step S2c) and notifies the terminal 210 of the completion of the setting (S2d). The sensing device 240 acquires sensing data (step S2e) and transmits it to the terminal 210 (step S2f). In step S2f, the acquired sensing data is abstracted, and the abstracted data is transmitted to the terminal 210.

In step S2g, the terminal 210 transmits a reception completion message to the sensing device 240. In step S2h, the abstracted data is added to the health condition information database 111. Steps S2b to S2h are repeated.

The health condition information database 111 is brought back to the home 100 (the terminal 210 transmits it to the home server 110) (step S2i).

Step S3: In step S3a, the home server 110 reflects the difference between the abstracted data of the health condition information database brought out and the health condition information database 111 brought back. In step S3b, the home server 110 deletes the health condition information database 111 of the terminal 210. In step S3c, the home server 110 acquires abstracted data from the sensing device 140 or the bi-function device 130 and stores the abstracted data in the health condition information database 111. The abstracted database is brought into the home server 310 of the residential accommodation 300 in the warm region (the home server 110 transmits the abstracted database to the home server 310) (step S3d).

Step S4: The home server 310 of the accommodation 300 notifies the home server 110 that the database 111 is being brought out in step S4a. In step S4b, the home server 310 acquires the abstracted data from the sensing device 340 or the bi-function home appliance 330 and adds the abstracted data to the health condition information database 111. The health condition information database 111 is brought back to home 100 (the home server 310 sends it to the home server 110) (step S4c).

Step S5: In step S5a, the home server 110 reflects the difference between the abstracted data of the health condition information database brought out and the health condition information database 111 brought back.

In the embodiment, an example of a hotel or a residential accommodation has been described as the non-daily living space, but the present invention is not limited to this, and the present invention may be applied to an in-vehicle space of a private car, a taxi, a train, or the like, an in-ship space of a private ship, a cruise ship, or the like, or an in-flight space of a private airplane, a passenger aircraft, or the like.

According to the embodiment, there are the following effects. (1) With the sensing device installed in the daily living space, it is possible to grasp the abstracted health condition of the resident (user) without consciousness. (2) In the case where the actuator device is directly controlled by the information of the sensing device, a special control system that requires a one-to-one relationship for each model or the number of devices is provided. In other words, when there is no one-to-one relationship between the sensing device and the actuator device, the control relationship cannot be maintained. On the other hand, when the actuator device supports health based on an abstracted health condition based on sensing data from the sensing device, a one-to-one relationship does not necessarily exist between the sensing device and the actuator device. As a result, even when a resident temporarily stays in a non-daily living space (e.g., private accommodation, hotel) or moves in a provisional living space composed of different actuator devices, health support can be provided by the actuator devices existing on the spot by carrying the abstracted health condition information. That is, by having the actuator device provide health support based on the abstracted health condition based on the sensing data from the sensing device, the versatility and portability of control by these devices are provided.

According to the above (1) and (2), the resident (user) can always receive health support without being conscious and regardless of the place and mode of the living space.

In the following, examples of abstractions of health conditions and the operation of actuator devices and the like based thereon will be described with reference to some embodiments.

### First Embodiment

Next, a description will be given of an example of determining the condition of a user's stomach in a toilet equipped with a toilet having a sensor.

Here, the toilet 140a of the home 100 is equipped with a toilet with a sensor, and includes a sensor for detecting the elevation condition of the toilet seat, a weight sensor for measuring the weight of the user on the toilet seat, a sensor for measuring the weight of the substance dropped in the toilet bowl, and a water contamination sensor for measuring the contamination of the water in the toilet bowl, and is configured to determine the excretion condition and the physical condition of the user by combining the measurement results of the various sensors. The toilet 240a of the hotel 200 includes a lower-level toilet bowl with few sensors, in which the weight sensor and the water contamination sensor of the toilet 140a are omitted. The toilet 340a of the residential 300 is equipped with a toilet of a higher model with built-in odor sensors and sensors in addition to the sensors of the toilet 140a. Hereinafter, when it is not necessary to distinguish between the toilets 140a, 240a, and 340a, the toilet is simply referred to as a toilet.

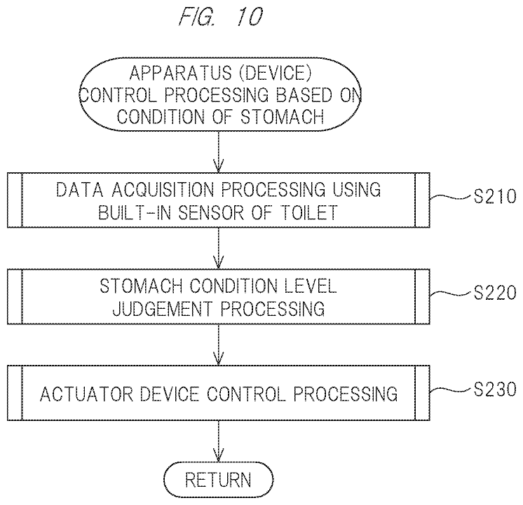

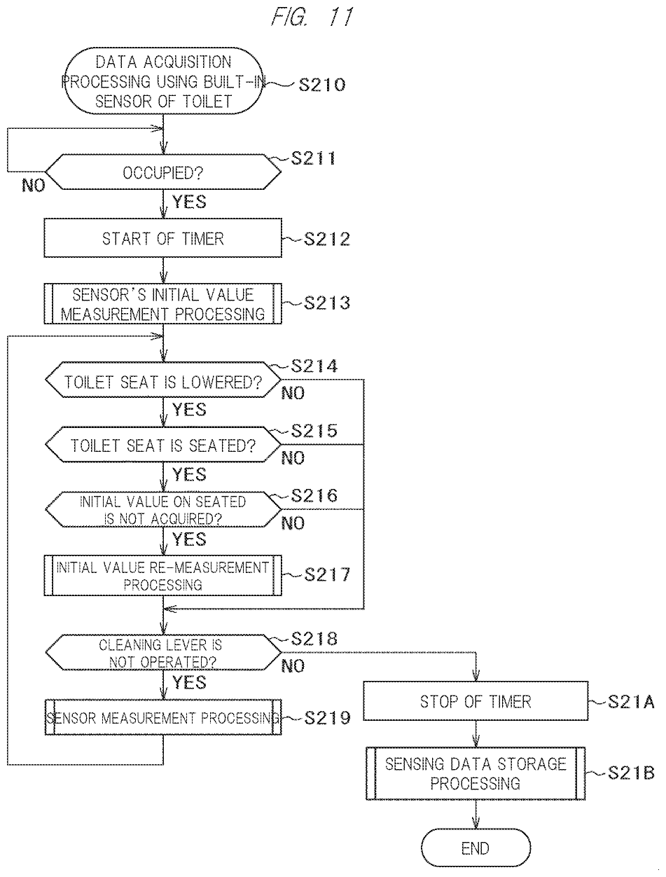

An outline of the device control processing by the condition of the stomach will be described with reference to FIG. 10. FIG. 10 is a flowchart of the device control processing based on the condition of the stomach. The device control processing by the stomach condition has the following steps.

In step S210, the sensing device 140 acquires sensing data, for example, the toilet 140a performs data acquisition processing using a built-in sensor.

In step S220, the analysis and abstraction of the sensing data, for example, the toilet 140a performs stomach condition level judgement processing (abstraction).

In step S230, control of the actuator device or the like by the abstracted data, for example, control processing of the actuator device 120 or the bi-function device 130 is performed.

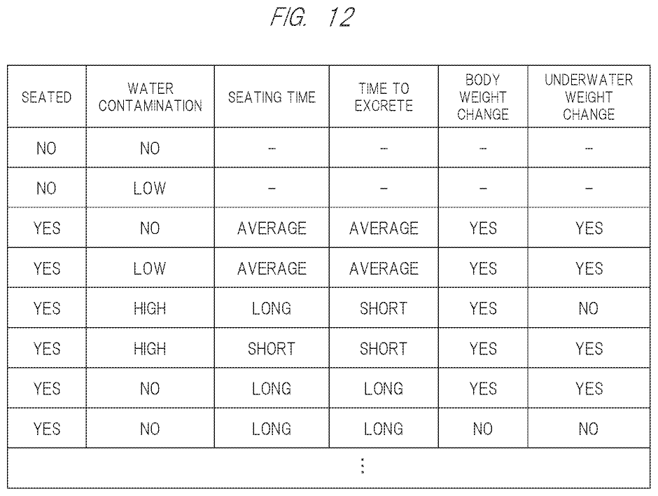

The data acquisition processing by the built-in sensor of the toilet (step S210) will be described with reference to FIGS. 11 and 12. FIG. 11 is a flowchart showing data acquisition processing by the toilet. FIG. 12 is a table showing possible combinations of sensing data by the toilet 140a.

First, the toilet determines whether the user enters the toilet or not (step S211), and when the user enters the toilet, a timer is started to measure the usage time (step S212). The toilet determines whether the washing lever is not operated (step S218), recognizes that the toilet has been discharged when the user operates the washing lever, stops the timer, and records the usage time (step S21A). The usage time may be calculated from the entry and exit times. In step S213, the various sensors measure and record initial values at the same time as the timer starts. After that, serial sensor measurements are performed (Step S219) and the excretion details are monitored until the cleaning lever operation.

When the washing lever is operated in a condition in which the toilet seat is raised (NO in step S214) (NO in step S218), the toilet performs storage processing of each sensing data (step S21B), compares the initial value of the water contamination sensor with the value at the time of monitoring, and when a small amount of contamination (turbidity) is detected, it is determined that the male has performed urination. Although the toilet 240ais not equipped with a water contamination sensor, it can be judged that excretion was performed from the normal operating time without detecting contamination.

When the toilet is in a condition in which the toilet seat is lowered (YES in step S214) and when the seating on the toilet seat is detected (YES in step S214), the various sensors recognize that the toilet seat is in the seated condition and start excretion processing, and measure the initial value again (step S217). When the cleaning lever is operated (NO in step S218), it is determined that the excretion is completed, and the storage processing of each sensing data is performed (step S21B). The toilet 140a compares the sensing data with the initial values of the various sensors and the values at the time of monitoring, and stores the data in the storage device 146 as the presence or absence of seating, the condition of water turbidity, the seating time, the time to discharge, the body weight change, and the underwater weight change as shown in FIG. 12.

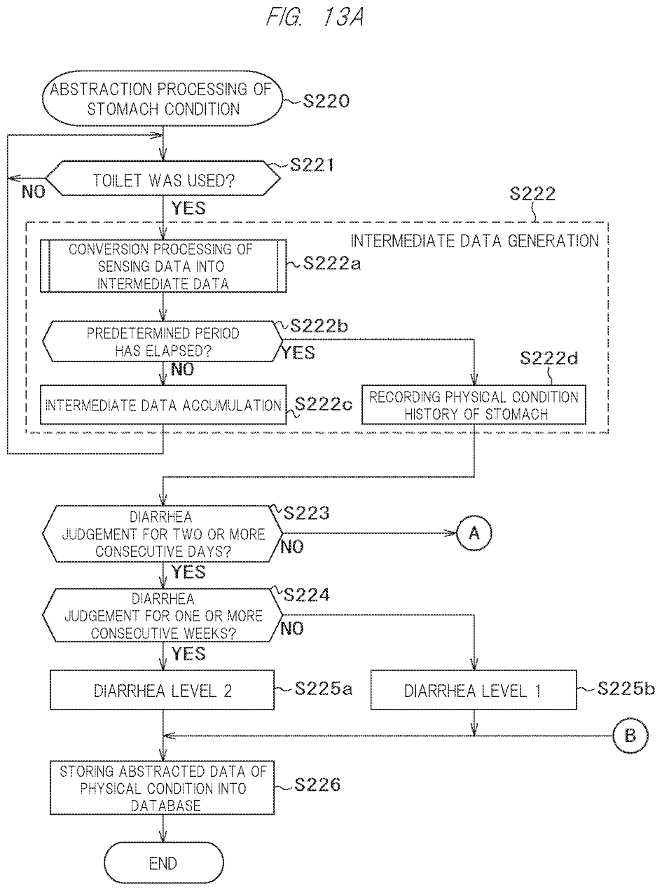

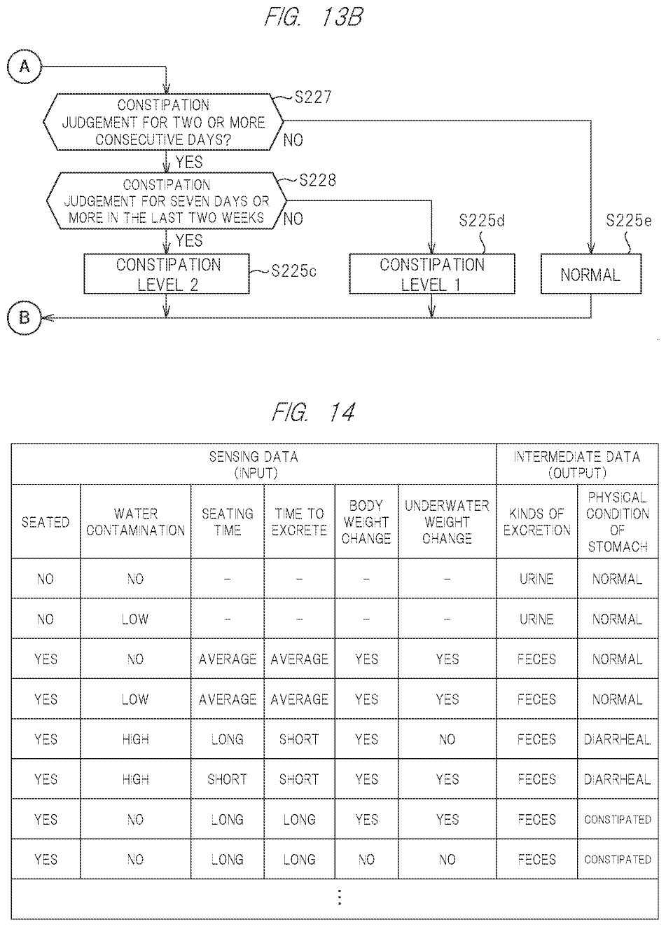

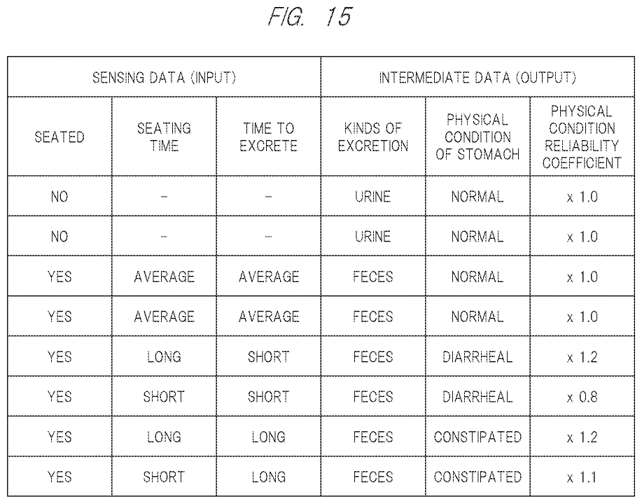

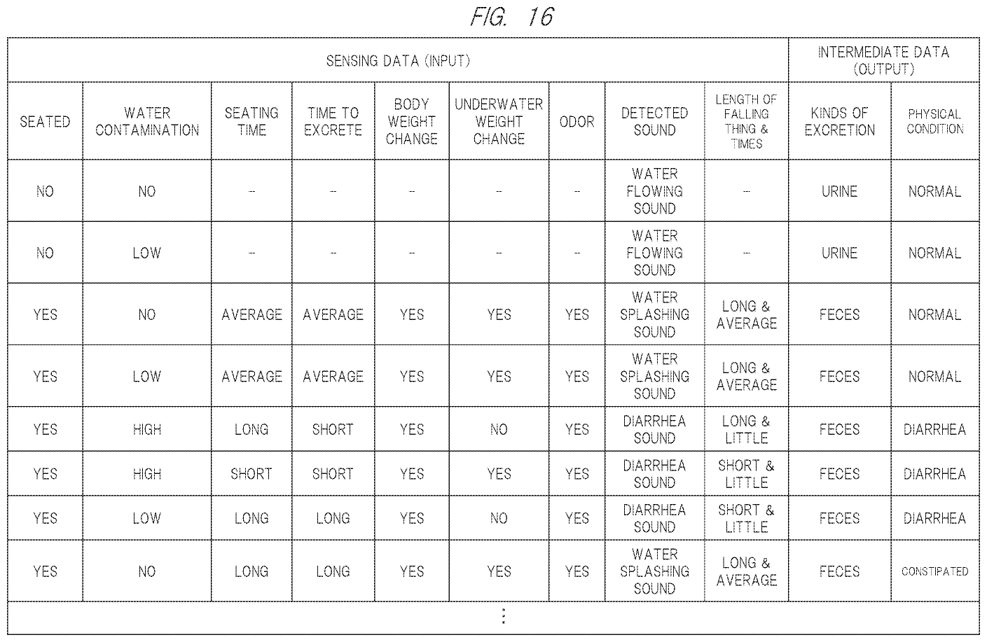

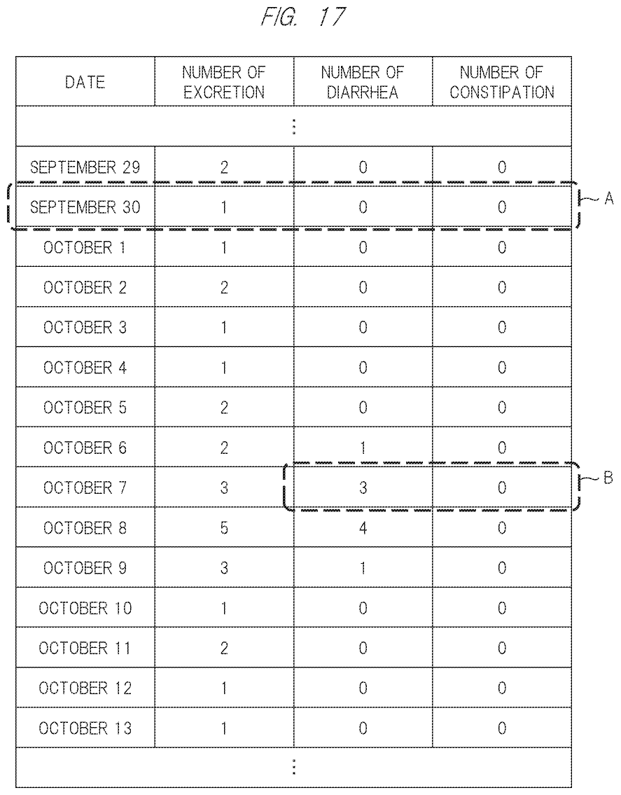

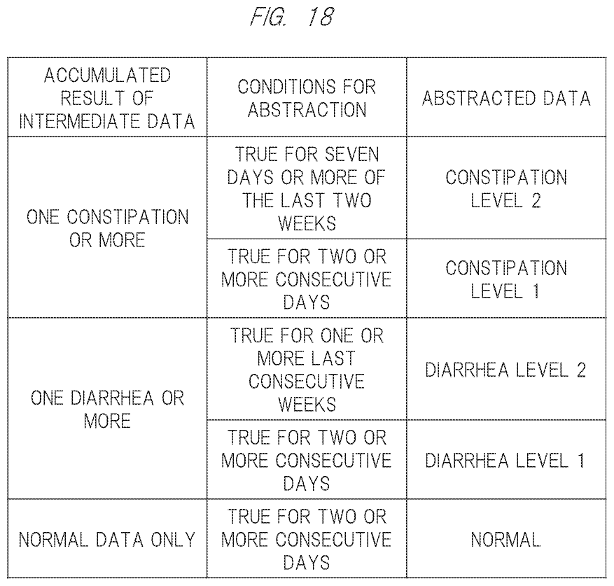

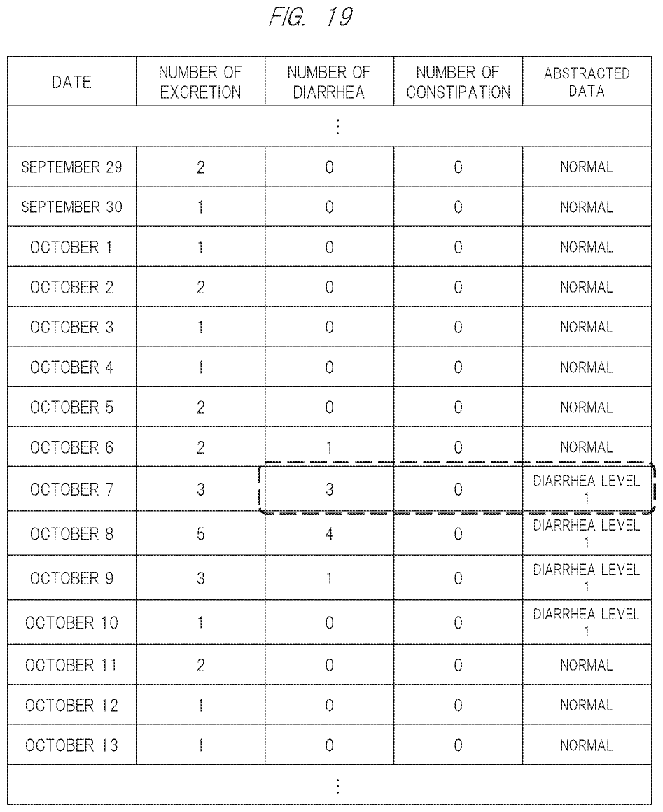

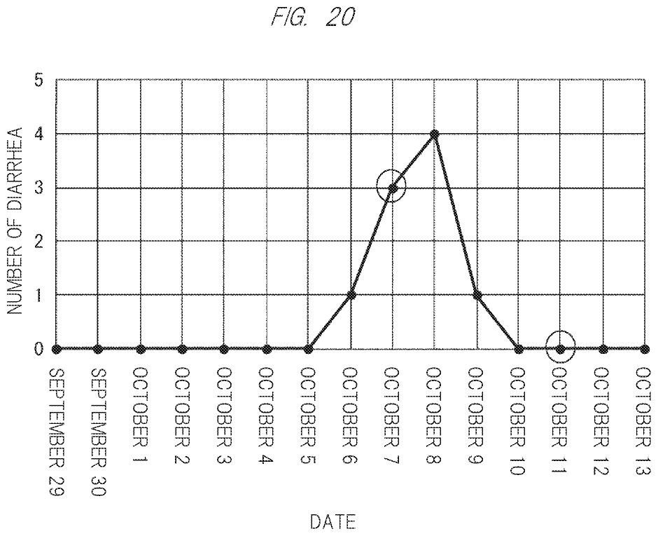

The abstraction processing of the stomach condition (the stomach condition level judgement processing) (step S220) will be described with reference to FIGS. 13A, 13B, and 14 to 20. FIGS. 13A and 13B are flowcharts of the abstraction processing of the stomach condition. FIG. 14 is a diagram showing a table for converting sensing data of the toilet 140a into intermediate data. FIG. 15 is a diagram showing a table for converting sensing data of the toilet 240a into intermediate data. FIG. 16 is a diagram showing a table for converting sensing data of the toilet 340a into intermediate data. FIG. 17 is a diagram showing a table in which the intermediate data is integrated day by day. FIG. 18 is a table showing conditions for abstracting the physical condition of the stomach. FIG. 19 is a diagram showing an example of a physical condition record of a stomach when diarrhea is detected in a certain period. FIG. 20 is a graph showing the number of times of diarrhea in FIG. 19 by a line graph.

The physical condition judgement processing for each use of the toilet is performed by abstraction processing of the stomach condition. The processing in the toilet 140a will be described below. The judgement processing may not be limited to the procedure of the example flowchart because the sensor configuration differs depending on the toilet facility, but the same processing is performed on the toilet 240a and the toilet 340a. Further, processing reduction for processing efficiency improvement and processing addition for high-performance improvement may be performed, and the judgement processing is not limited to an example.

In Step S222, the toilet 140a determines whether the toilet was used or not (Step S221), and when used, the intermediate data is generated based on the sensing data (FIG. 12) obtained in Step S210. In step S222a, processing for converting the sensing data into intermediate data is performed using the conversion table shown in FIG. 14. In order to judge the condition of the user's stomach from the measurement results of the various sensors, an intermediate data conversion table (FIGS. 14 to 16) as a physical condition judgement table for each toilet use is used to select one that matches the data. The combination of sensing data determines whether the stomach condition is normal, diarrheal, or constipated.

As shown in FIG. 15, since the number of sensors in the toilet 240a is smaller than that in the toilet 140a, the number of items used for the judgement tends to be reduced.

Therefore, the measurement result is multiplied by the physical condition reliability coefficient to improve the accuracy of the physical condition judgement. As shown in FIG. 16, since the number of sensors in the toilet 340a is larger than that in the toilet 140a, the accuracy of the physical condition judgement is improved.

The physical condition judgement table is not limited to an exemplary combination, and items may be changed by sensors mounted on a toilet bowl, other physical conditions may be added, or items may be updated using communication means. In addition, the physical condition judgement table may indicate the condition of the stomach as a physical condition point, may determine the diarrhea feeling as +1 and the constipation feeling as −1, or may determine the assignment to the program flag. In the case of judging by the physical condition point, for example, a point other than 0 is judged as the physical condition abnormality, but it is also conceivable that it is always other than 0 depending on the physical condition of the individual. In this case, the period is not limited, but the accumulated data for a long period may be used as the reference data for determining the physical condition of the user, and the physical condition may be determined from the separation from the daily recording. Further, a system in which the physical condition of the user is inferred from the daily use situation without preparing a physical condition judgement table may be used.

Next, the toilet 140a determines whether a predetermined period (e.g., one day) has elapsed or not (Step S222b), and if a predetermined period has not elapsed, the number of stools, diarrhea, and constipation are accumulated (Step S222c). When the predetermined period has elapsed, it is recorded in the physical condition history of the stomach as shown in FIG. 17 in step S222d. In the table of FIG. 17, the judgement frequency of each physical condition data is recorded as a history. For example, the portion surrounded by the broken line A is the daily bowel movement information accumulated in the step S222c, and the portion surrounded by the broken line B is the accumulated number of bowel movement situations of one day (accumulation of the intermediate data).

Next, the toilet 140a generates abstracted data based on the abstraction condition of the intermediate data as shown in FIG. 18. FIG. 18 is a diagram for determining the physical condition as abstracted data when the physical condition frequency of the history meets a certain condition. The period, such as the number of days of judgement, can be changed according to symptoms and individual differences. For example, it is determined whether or not there is diarrhea judgement for two or more consecutive days (step S223), in the case of YES, it is determined whether or not there is diarrhea judgement for one or more consecutive weeks, and in the case of YES, “diarrhea level 2” is generated as abstracted data (step S225a). If NO in step S224, the toilet 140a generates “diarrhea level 1” as the abstracted data (step S225b).

In the case of NO in step S223, it is determined whether or not there is a constipation judgement for two consecutive days or more (step S227), in the case of YES, it is determined whether or not there is a constipation judgement for seven days or more of the last two weeks (step S228), and in the case of YES, “constipation level 2” is generated as abstracted data (step S225c). If NO in step S228, the toilet 140 a generates “constipation level 1” as the abstracted data (step S225d). If NO in step S227, the toilet 140 a generates “normal” as the abstracted data (step S225e).

As shown in FIGS. 19 and 20, the number of times of diarrhea is 0 until October 5, indicating that the resident is comfortable. On the other hand, the number of times of diarrhea became one or more from October 6, indicating that the resident was diarrheal.

No particular notification is given during periods of no abnormal physical condition, but the toilet transmits health condition information (abstracted data) to the home server 110 on October 7, when the number of days of diarrhea is one or more (the abstracted data becomes “diarrhea level 1” when diarrhea is detected for two consecutive days) on the second day. Having received the abstracted data, the home server 110 transmits health condition information and control signals to the actuator device 120 or the bi-function device 130. This information transmission continues until October 11, when the average of the intermediate data continues for two or more days (the abstracted data is “normal”), and the actuator device 120 or the bi-function device 130 performs its own control so as to be comfortable as much as possible in accordance with the physical condition of the user, or proposes a meal or an action to the user in order to restore the physical condition.

Although the physical condition judgement is performed from the daily recording in this embodiment, it is also possible to use the toilet usage information status for each hour, for example, to predict a physical condition change such as a sign of a physical condition change when the number of times of use increases, and to notify the user of the change in physical condition at an early stage.

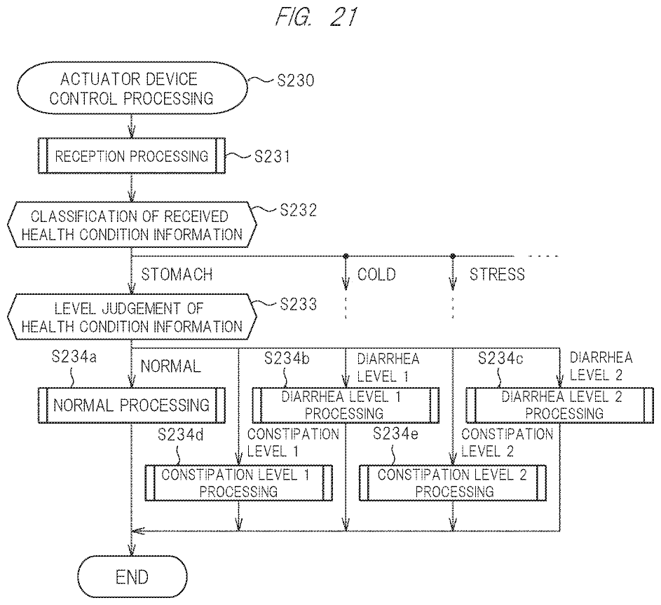

FIG. 21 is a flowchart showing the operation of the actuator device or the like at home. When the actuator device 120 receives abstracted data from the home server 110, it selects and controls the optimum processing from the classification of health conditions and its level. This processing is similarly applied to the bi-function device 130. The same processing is performed even when the actuator system 220 and the bi-function device 230 receive the abstracted data from the terminal 210, and the same processing is performed even when the actuator device 320 and the bi-function device 330 receive the abstracted data from the home server 310.

The actuator device 120 performs reception processing (step S231), and the received health condition information (abstracted data) determines the classification of the stomach, cold, stress, and the like (step S232). In the case of a stomach, the level of the health condition information (abstracted data) is determined (step S233). In the case of “normal”, the following normal processing is performed (step S234a), in the case of “diarrhea level 1”, the following diarrhea level 1 processing is performed (step S234b), in the case of “diarrhea level 2”, the following diarrhea level 2 processing is performed (step S234c), in the case of “constipation level 1”, the following constipation level processing is performed (step S234d), and in the case of “constipation level 2”, the following constipation level 2 processing is performed (step S234e).

Note that steps S231 to S233 may be performed by the home server 110, and steps S234a to S234e may be performed by the actuator device 120 or the bi-function device 130.

In the processing of step S234 a during normal operation, for example, the air conditioner 130a performs normal operation. The refrigerator 120e proposes, for example, “Consider a menu made of materials in the compartment” in the voice guidance. When standing in front of the washstand 130b, the washstand 130b announces, e.g., “It's still pleasant today,” by voice guidance. In addition, the washstand 130b displays a health symbol on the built-in display.

In the processing of the diarrhea level 1 in step S234b, for example, the air conditioner 130a raises the set temperature by 1° C. so that the stomach does not cool. Refrigerator 120e suggests, for example, in a voice guidance, “Is your stomach sick? Let's refill with sports drinks?” Standing in front of the washbasin 130b, the washbasin 130b offers advice on improving physical condition by telling the resident by the voice guidance that you are diarrhea-prone, for example: “it feels like your stomach is getting down. Let's have a good meal for hydration and digestion.” In addition, the washstand 130b displays a body symbol on the built-in display and highlights the stomach portion of the body symbol by flashing or the like, thereby making it possible to confirm the physical condition at a glance.

In the processing of the diarrhea level 2 in step S234c, for example, the air conditioner 130a operates in the same manner as the diarrhea level 1. Refrigerator 120e suggests, for example, “You are sick about your stomach. Consider a good menu for digestion,” in a voice guidance. When standing in front of the washstand 130b, the washstand 130b provides voice guidance saying, for example, “Diarrhea is prolonged. Consider consultation with your doctor.” In addition, the washstand 130b displays a body symbol on the built-in display and highlights the stomach portion of the body symbol by flashing or the like.

In the processing of the constipation level 1 in step S234d, for example, the air conditioner 130a operates to direct the louver downward to warm the foot in order to be relieved or to prevent the constipation from being deteriorated due to the cooling of the foot. The refrigerator 120e proposes, for example, “Is your stomach tight? Should you take a lot of vegetables and fruits?” in the voice guidance, that you can consume dietary fiber, such as vegetables and fruits, if you have a tight stomach. Standing in front of the washbasin 130b, the washbasin 130b provides by audio guidance, for example, saying, “You feel constipated. Let's have a diet containing fluids and dietary fiber,” and proposing advice for improving physical condition. In addition, the washstand 130b cautioned with a voice guidance, saying, “If you step on for a long time, you will develop hemorrhoids.” In addition, the washstand 130b displays a body symbol on the built-in display and highlights the stomach area by blinking or the like, thereby making it possible to confirm the bad physical condition at a glance. The rice cooker 120d makes a voice guidance suggesting “constipation? How about pruritus?”

In the processing of constipation level 2 in step S234d, for example, the air conditioner 130a increases the set temperature by 1° C. in addition to the operation of “constipation level 1”. The refrigerator 120e suggests, for example, in a voice guidance, “You're hungry. Consider a menu that allows you to pick up dietary fiber.” When standing in front of the washstand 130b, the washstand 130b provides voice guidance, e.g., “Consult your doctor because constipation is prolonged.” The washstand 130b displays a body symbol on the built-in display and highlights the stomach area by blinking or the like, similarly to the “constipation level 1”.

Hotel 200 differs from Hotel 100 in terms of equipment configuration and equipment function, so even information about the same physical condition differs in operation. For example, the refrigerator 220e is not normally provided with foodstuffs. Therefore, proposals are made to encourage meals in the hotel cafeteria and cafes. Since the washstand 230b does not have a display function, only voice guidance like that of the washstand 130b is performed. Since the air conditioner 230a has the same function as that of the air conditioner 130a, the same operation as that of the air conditioner 130a is performed.

For example, in the normal processing of step S234a, the refrigerator 220e suggests in the voice guidance “What about the recommended meal for the hotel cafeteria today?”

In the processing of diarrhea level 1 in step S234b, the refrigerator 220e suggests in a voice guidance that “Is your stomach sick? Let's refill with sports drinks. The hotel's store is in front of the 1F lobby?”

In the processing of the diarrhea level 2 in the step S234c, the refrigerator 220e notifies in a voice guidance that “You are bad about your stomach. We have prepared a digestible food in this hotel cafeteria.”

The processing of constipation level 1 in step S234d proposes as follows: “Do you feel hungry? Take a lot of vegetables and fruits. We have prepared a snack at this hotel cafe.”

The processing of constipation level 2 in step S234e says, “You are hungry. I have prepared a good food in the hotel cafeteria to encourage you to have a good meal.”

### Second Embodiment

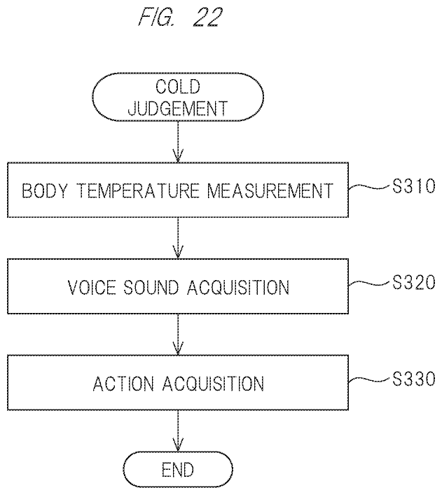

An example of the cold judgement will be described with reference to FIGS. 22 to 37. FIG. 22 is a flowchart showing an outline of the cold judgement.

This example is an example of processing for detecting a difference between a normal condition and a cold and determining it as a cold by sensing and signal processing (steps S310 to S330) of body temperature, utterance content (nasal closing sound, cough, sneezing, nasal sucking sound, sneezing sound), and action (sneezing, sneezing motion) of a user who is a resident.

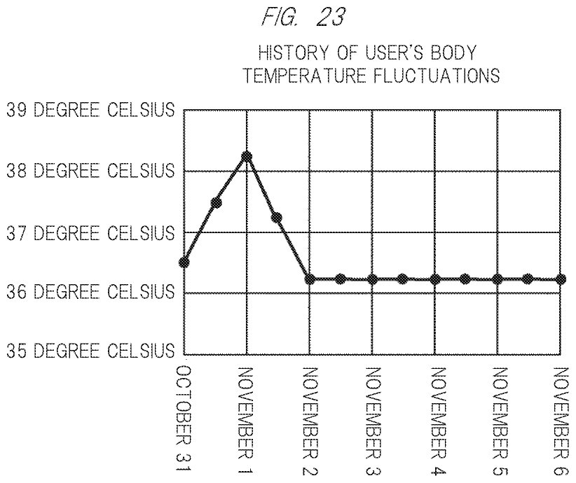

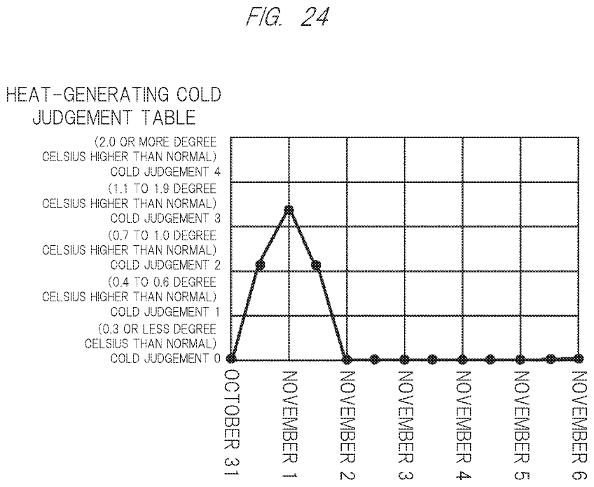

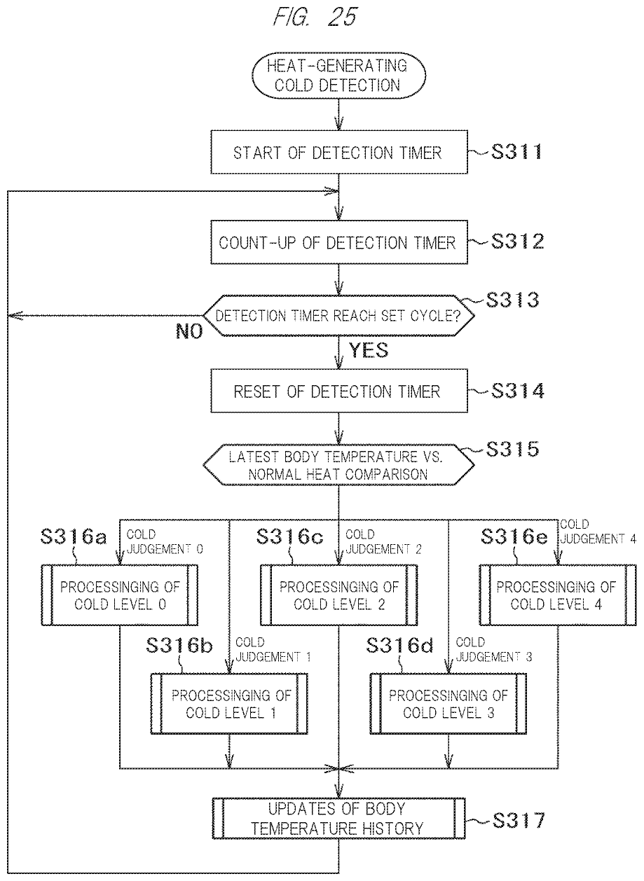

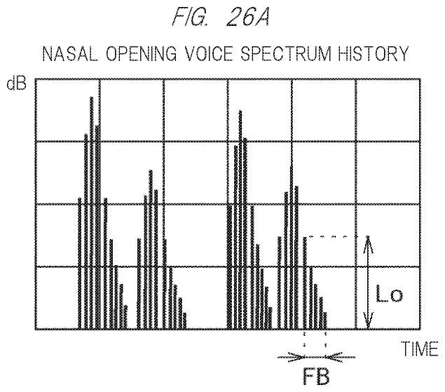

The body temperature of the user is measured by a temperature sensor or the like incorporated in the sensing device 140 or the bi-function device 130. The device continuously and periodically records the user's body temperature fluctuations and accumulates a history of the user's body temperature fluctuations as shown in FIG. 23. Although the body temperature is recorded every half day in FIG. 23, the measurement interval may be shortened in order to more accurately detect the body temperature fluctuation. In addition, it is assumed that the user's normal heat is calculated in advance from the history of the body temperature fluctuation. FIG. 24 is a diagram showing a heat-generating cold judgement table for converting a user's body temperature to a cold level. FIG. 25 is a flowchart of the heat-generating cold detection processing.

After the body temperature is measured, the sensing device 140 or the bi-function device 130 starts a detection timer (step S311) and counts the detection timer (step S312). It is determined whether the detection timer has reached the set cycle or not, and if YES, the detection timer is reset in step S314. In step S315, the user's body temperature is periodically detected, and whenever the set period elapses, a cold judgement is performed. In this example, the body temperature rise amount (the difference between the latest body temperature measurement value of the user and the normal heat information) is classified into five abstracted cold judgements (abstracted data) as shown in FIG. 24.

The cold judgement 0 is a condition in which the measured body temperature is 0 to 0.3° C. higher than the normal heat, and when the cold judgement 0 is determined in step S315, processing of the cold level 0 is performed in step S316a.

The cold judgement 1 is a condition in which the measured body temperature is higher than the normal heat by 0.4 to 0.6° C., and when the cold judgement 1 is determined in step S315, processing of the cold level 1 is performed in step S316b.

The cold judgement 2 is a condition in which the measured body temperature is higher than the normal heat by 0.7 to 1.0° C., and when the cold judgement 2 is determined in step S315, processing of the cold level 2 is performed in step S316c.

The cold judgement 3 is a condition in which the measured body temperature is 1.1 to 1.9° C. higher than the normal heat, and when the cold judgement 3 is determined in step S315, processing of the cold level 3 is performed in step S316d.

The cold judgement 4 is a condition in which the measured body temperature is higher than the normal heat by 2.0° C. or more, and when the cold judgement 4 is determined in step S315, processing of the cold level 4 is performed in step S316e.

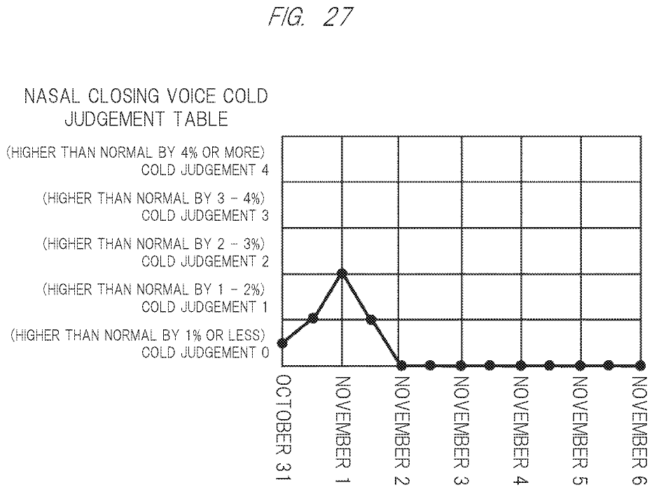

As shown in FIG. 27, the change in the user's body temperature in FIG. 25 is determined as cold judgement 2 for the second time on October 31, cold judgement 3 for the first time on November 1, and cold judgement 2 for the second time on November 1.

In step S317, the body temperature history is updated, and the processing returns to step S312.

The classification of the health condition may be performed by using the absolute temperature of the body temperature as a threshold value and classifying the health condition. Temporary increases in body temperature due to exercise, bathing, excessive air conditioning, etc. are also conceivable. In order to distinguish them from each other, a processing may be added in which, when the body temperature increase amount is maintained continuously for one hour or more, the processing shifts to the judgement of a febrile cold, and when the body temperature returns to a flat temperature within one hour, the judgement of a cold is 0 (healthy).

Methods of judging a cold from utterance include, for example, a cold judgement by a nasal closing voice, a cold judgement by a cough, a sneezing, a nasal sucker, and a cold judgement by a collapsing nose.

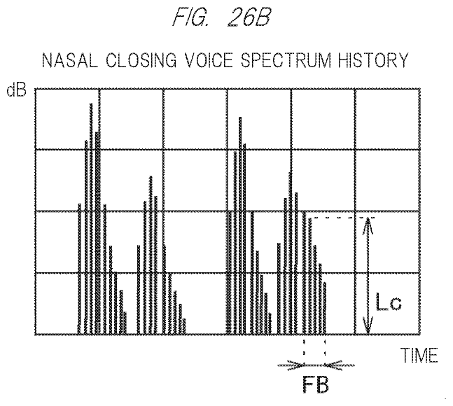

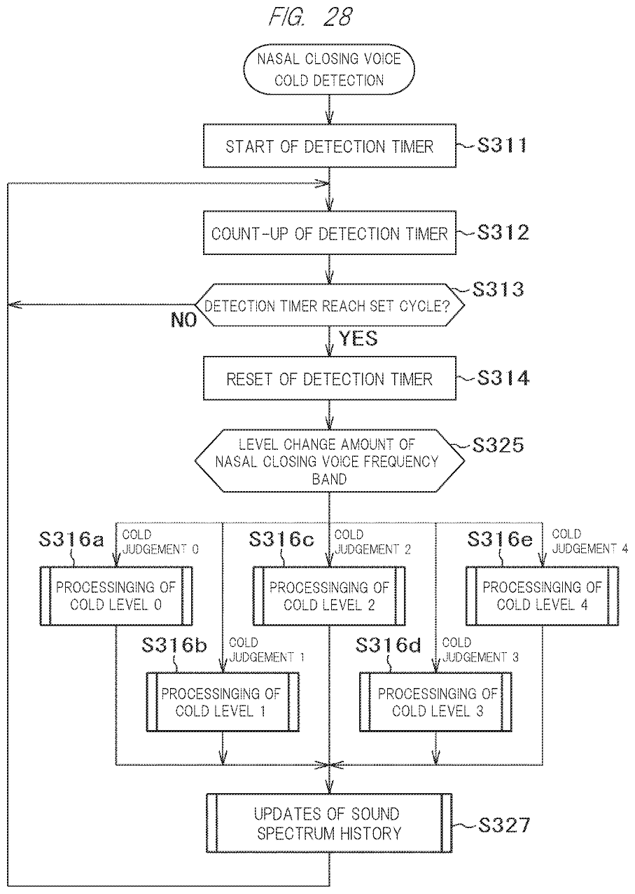

First, the cold judgement by the nasal closing voice will be described with reference to FIGS. 26A, 26B, 27, and 28. FIG. 26A is a diagram showing a user's nasal opening voice spectrum history, and FIG. 26B is a diagram showing a nasal closing voice spectrum history. FIG. 27 is a diagram showing an example of a nasal closing cold judgement table. FIG. 28 is a flowchart of the nasal closing cold detection processing.

Using a microphone incorporated in the sensing device 140, a normal voice (nasal opening voice) spectrum history of the user as shown in FIG. 26A is stored, the degree of the nasal voice (nasal closing voice) changed by the cold is numerically converted from the amount of change (Lc-Lo) in the level of the partial frequency band (FB) of the user voice spectrum as shown in FIG. 26B, and a nasal closing voice cold judgement is performed using the level of the cold caused by the nasal running and nasal blockage as abstracted data. The level of nasal closing voice (Lc) is greater than the level of nasal opening voice (Lo).

After the voice spectrum is measured, the sensing device 140 or the bi-function device 130 starts a detection timer (step S311) and counts the detection timer (step S312). It is determined whether the detection timer has reached the set cycle or not (step S313), and in the case of YES, the detection timer is reset (step S314). When a predetermined period has elapsed, a judgement of a cold is made by detecting a nasal closing voice in step S325. In this example, the voice spectrum is classified into five abstracted cold judgements as shown in FIG. 27 based on the level change amount of the nasal closing voice frequency band (difference between the level of the nasal closing voice and the level of the nasal opening voice).

The cold judgement 0 is a condition in which the level of the measured nasal closing voice is higher than the normal nasal opening voice by 0% or more and less than 1%, and when the cold judgement 0 is determined in step S325, processing of the cold level 0 is performed in step S316a.

The cold judgement 1 is a condition in which the level of the measured nasal closing voice is higher than that of the normal nasal opening voice by 1% or more and less than 2%, and when the cold judgement 1 is determined in step S325, processing of the cold level 1 is performed in step S316b.

The cold judgement 2 is a condition in which the level of the measured nasal closing voice is higher than that of the normal nasal opening voice by 2% or more and less than 3%, and when the cold judgement 2 is determined in step S325, processing of the cold level 2 is performed in step S316c.

The cold judgement 3 is a condition in which the level of the measured nasal closing voice is higher than that of the normal nasal opening voice by 3% or more and less than 4%, and when the cold judgement 3 is determined in step S325, processing of the cold level 3 is performed in step S316d.

The cold judgement 4 is a condition in which the level of the measured nasal closing voice is higher than that of the normal nasal opening voice by 4% or more, and when the cold judgement 4 is determined in step S325, processing of the cold level 4 is performed in step S316e.

As shown in FIG. 27, the second cold judgement on October 31 is 1, the first cold judgement on November 1 is 2, and the second cold judgement on November 1 is 1.

In step S327, the voice spectrum history is updated, and the processing returns to step S312.

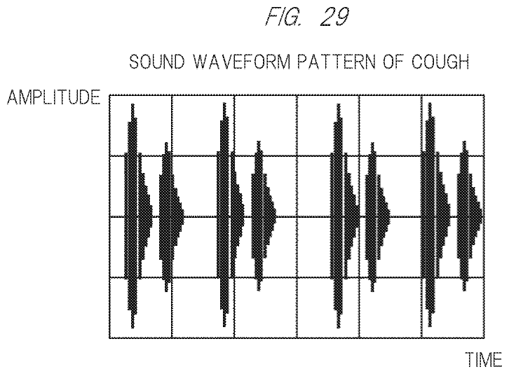

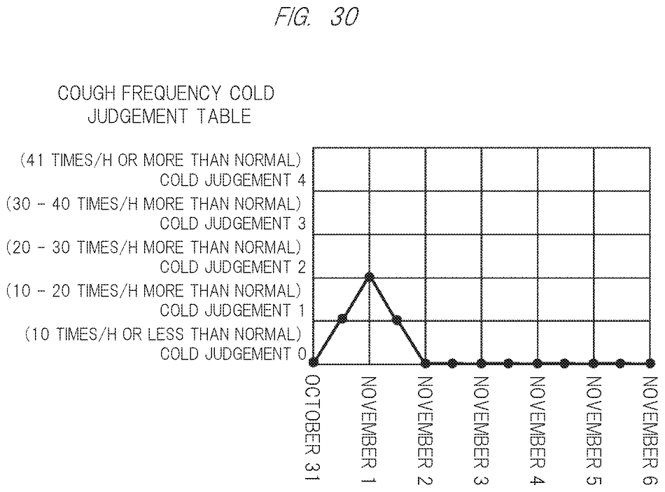

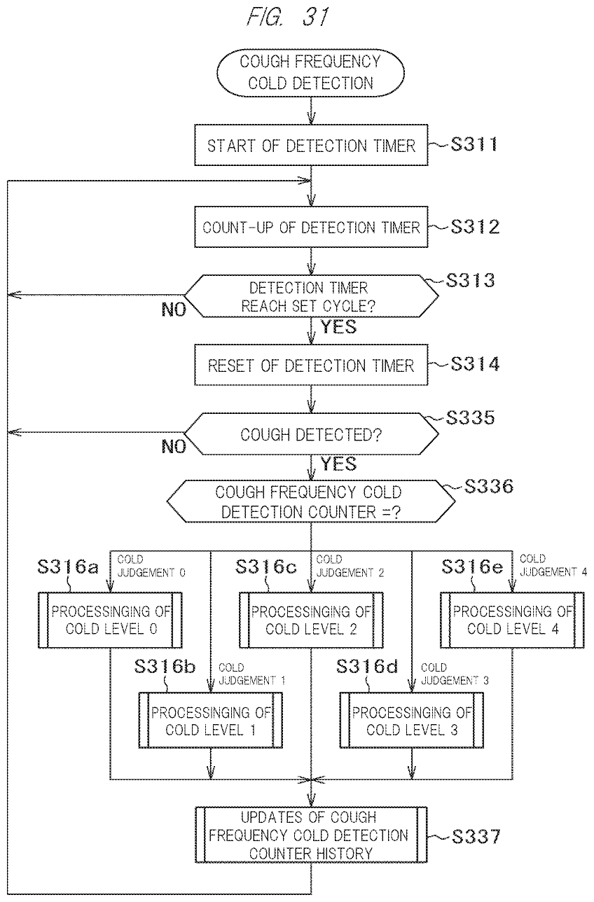

Next, cold judgement by cough will be described with reference to FIGS. 29 to 31. FIG. 29 is a diagram showing a sound waveform pattern of a cough of a user. FIG. 30 is a diagram showing an example of a cough frequency cold judgement table. FIG. 31 is a flowchart of cough frequency cold detection processing.

The sound waveform pattern of the cough of the user as shown in FIG. 29 is detected using a microphone built in the sensing device 140, and the history of the number of coughs is stored. Cough frequency cold judgement is carried out using the level of the cold symptom as abstracted data from the amount of change with the frequency of the cough at normal condition.

After the sound waveform pattern is measured, the sensing device 140 or the bi-function device 130 starts a detection timer (step S311) and counts the detection timer (step S312). It is determined whether the detection timer has reached the set cycle or not (step S313), and in the case of YES, the detection timer is reset (step S314). After a predetermined period has elapsed, a judgement of a cold due to cough is made in steps S335 and S336. In this example, the cough is detected from the sound waveform pattern (step S335), and the number of coughs is accumulated by the cough frequency cold detection counter, and from the difference between the measured cough frequency and the normal cough frequency, as shown in FIG. 30, the cough is classified into five abstracted cold judgements (step S336).

The cold judgement 0 is a condition in which the measured cough frequency is greater than or equal to 0 and less than 10 times with respect to the cough frequency in the normal condition, and when the cold judgement 0 is determined in step S336, processing of the cold level 0 is performed in step S316a.

The cold judgement 1 is a condition in which the measured cough frequency is 10 or more and less than 20 times as compared with the cough frequency in the normal condition, and when the cold judgement 1 is judged in step S336, the processing of the cold level 1 is performed in step S316b.

The cold judgement 2 is a condition in which the measured number of coughs is 20 or more and less than 30 times as compared with the normal number of coughs, and when the cold judgement 2 is determined in step S336, processing of the cold level 2 is performed in step S316c.

The cold judgement 3 is a condition in which the number of coughs measured is 30 or more and less than 40 times as compared with the number of coughs in the normal condition, and when the number of coughs is determined to be the cold judgement 3 in step S336, processing of the cold level 3 is performed in step S316d.

The cold judgement 4 is a condition in which the number of coughs measured is 41 or more times higher than the number of coughs in the normal condition, and when the cold judgement 4 is determined in step S336, the judgement processing of the cold level 4 is performed in step S316e. As shown in FIG. 30, the second cold judgement on October 31 is 1, the first cold judgement on November 1 is 2, and the second cold judgement on November 1 is 1.

The history of the cough frequency cold detection counter is updated in step S337, and the processing returns to step S312.

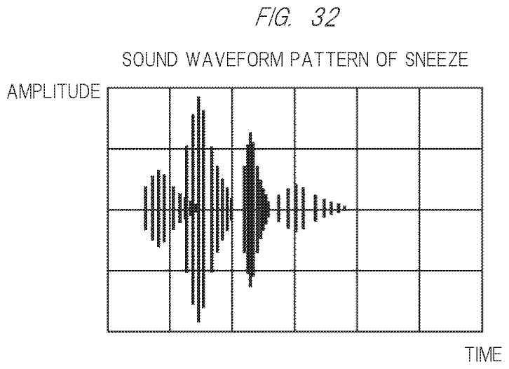

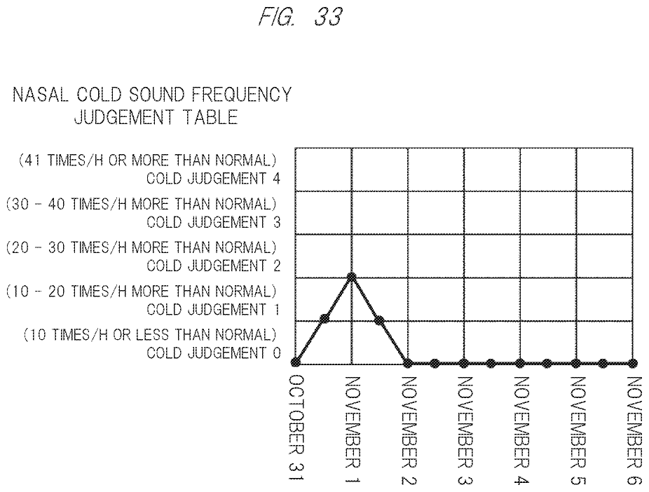

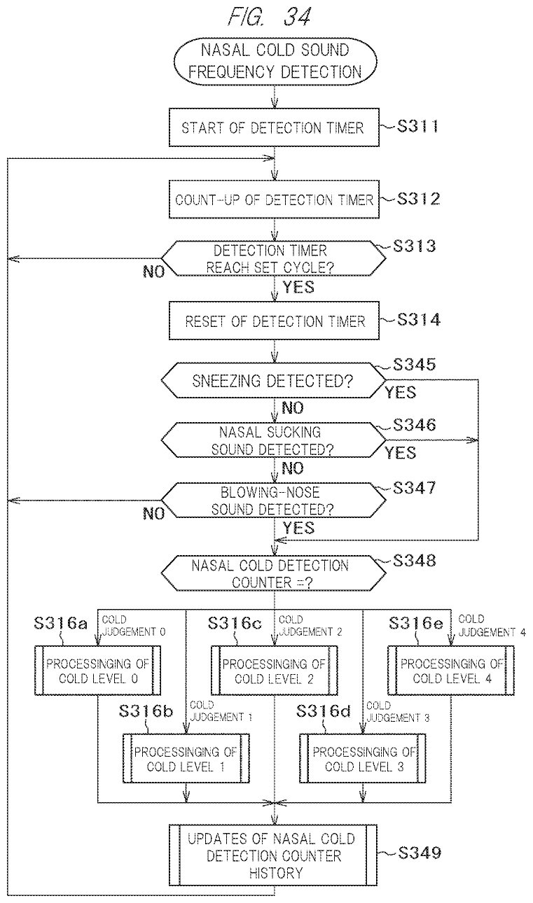

Next, a judgement of a nasal cold by sneezing, a nasal suck, and a collapsing nose will be described with reference to FIGS. 32 to 34. FIG. 32 is a diagram showing a sound waveform pattern of the sneezing of the user. FIG. 33 is a diagram showing an example of a nasal cold sound frequency judgement table. FIG. 34 is a flowchart of the nasal cold sound frequency detection processing.

The sound waveform pattern of the user's sneezing as shown in FIG. 32 is detected using a microphone built in the sensing device 140, and the history of the number of times is stored. Similarly, a history of the number of times of the user's nasal sounds (sounds that suck the nose) and bowel sounds (sounds that chew the nose) is stored, and a judgement of the number of times of nasal cold sounds is performed using the level of cold symptoms as abstracted data based on the amount of change from the normal condition. The number of sneezing, nasal sucking, and gynasal sounds is referred to as the number of nasal cold sounds.

After the sound waveform pattern is measured, the sensing device 140 or the bi-function device 130 starts a detection timer (step S311) and counts the detection timer (step S312). It is determined whether the detection timer has reached the set cycle or not (step S313), and in the case of YES, the detection timer is reset (step S314). When a predetermined period has elapsed, a cold is judged based on the sound waveform pattern in steps S345 to S347. In this example, sneezing is detected from the sound waveform pattern (step S345), or a nasal sucking sound is detected (step S346), or a sneezing sound is detected (step S347). The number of times is accumulated by the nasal cold detection counter, and the difference between the number of measured sneezes and the number of times of sneezing in the normal condition, or the difference between the number of times of measured sneezing and the number of times of normal sneezing, or the difference between the number of times of measured sneezing and the number of times of normal sneezing are classified into five abstracted cold judgements as shown in FIG. 33 (step S348).

The cold judgement 0 is a condition in which the measured number of times of the nasal cold sound is greater than the number of times of the nasal cold sound in the normal condition by 0 or more and less than 10 times, and when the cold judgement 0 is determined in step S348, processing of the cold level 0 is performed in step S316a.

The cold judgement 1 is a condition in which the measured number of times of the nasal cold sound is 10 or more and less than 20 times as compared with the number of times of the nasal cold sound in the normal condition, and when the cold judgement 1 is determined in step S348, processing of the cold level 1 is performed in step S316b.

The cold judgement 2 is a condition in which the measured number of times of the nasal cold sound is 20 or more and less than 30 times as compared with the number of times of the nasal cold sound in the normal condition, and when the cold judgement 2 is determined in step S348, processing of the cold level 2 is performed in step S316c.

The cold judgement 3 is a condition in which the measured number of times of the nasal cold sound is 30 or more and less than 40 times as compared with the number of times of the nasal cold sound in the normal condition, and when the cold judgement 3 is determined in step S348, processing of the cold level 3 is performed in step S316d.

The cold judgement 4 is a condition in which the number of measured nasal cold sounds is 41 or more times larger than the number of normal nasal cold sounds, and when the cold judgement 4 is determined in step S348, processing of the cold level 4 is performed in step S316e.

As shown in FIG. 33, the second cold judgement on October 31 is 1, the first cold judgement on November 1 is 2, and the second cold judgement on November 1 is 1.

In step S349, the history of the nasal cold detection counter is updated, and the processing returns to step S312.

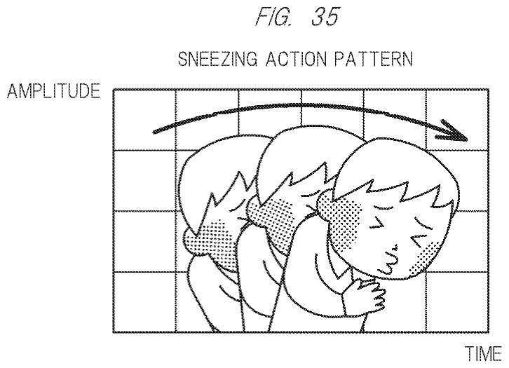

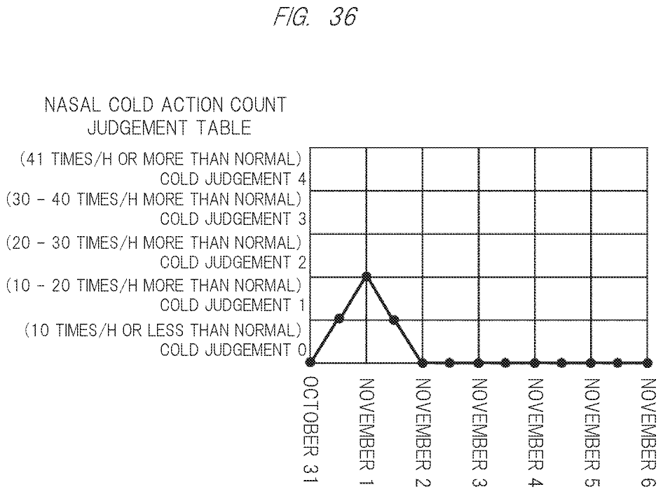

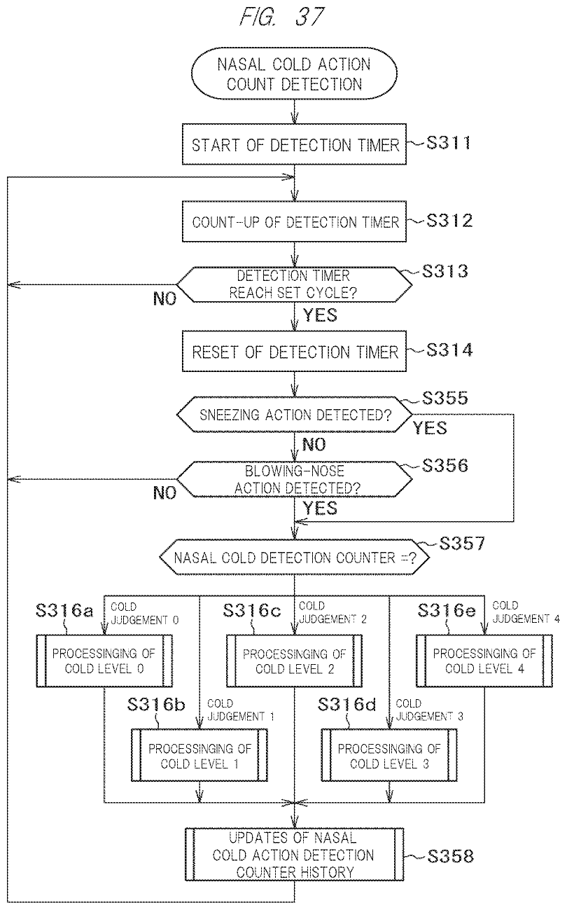

The cold judgement by the operation accompanying the nasal cold will be described with reference to FIGS. 35 to 37. FIG. 35 is a diagram showing a sneezing action pattern of a user. FIG. 36 is a diagram showing an example of a nasal cold action count judgement table. FIG. 37 is a flowchart of a processing of detecting the number of nasal cold actions.

The user's sneezing action pattern as shown in FIG. 35 is detected using a movie camera built in the sensing device 140, and the history of the number of times is stored. In the same manner, a history of the number of times of the user's collateral action (the action of blowing the nose) is stored, and the number of times of the nasal cold action is determined using the level of the symptoms of the cold as abstract data from the amount of change from the normal condition.

The sensing device 140 or the bi-function device 130 starts the detection timer (step S311) after measuring the operation of the nasal cold and counts the detection timer (step S312). It is determined whether the detection timer has reached the set cycle or not (step S313), and in the case of YES, the detection timer is reset (step S314). When a predetermined period has elapsed, it is determined whether the cold is caused by the nasal cold action in steps S355 to S357. In this example, the sneezing action is detected from the user's action (step S355) or the blowing action is detected (step S356), and the number of times is accumulated by the sneezing action detection counter, and the difference between the measured number of sneezing actions and the normal number of sneezing actions, or the difference between the measured number of sneezing actions and the normal number of sneezing actions is classified into five abstracted cold decisions as shown in FIG. 36 (step S357).

The cold judgement 0 is a condition in which the measured number of times of the nasal cold action is greater than or equal to 0 and less than 10 times than the number of times of the nasal cold action in the normal condition, and when the cold judgement 0 is determined in step S357, processing of the cold level 0 is performed in step S316a.

The cold judgement 1 is a condition in which the measured number of times of the nasal cold action is 10 or more and less than 20 times as compared with the number of times of the nasal cold sound in the normal condition, and when the cold judgement 1 is judged in the step S357, the processing of the cold level 1 is performed in a step S316b.

The cold judgement 2 is a condition in which the measured number of nasal cold actions is 20 or more and less than 30 times as compared with the number of nasal cold actions in the normal condition, and when the cold judgement 2 is determined in step S357, processing of the cold level 2 is performed in step S316c.

The cold judgement 3 is a condition in which the measured number of times of the nasal cold action is 30 or more and less than 40 times as compared with the number of times of the nasal cold action in the normal condition, and when the cold judgement 3 is judged in the step S357, the processing of the cold level 3 is performed (step S316d).

The cold judgement 4 is a condition in which the measured number of nasal cold actions is 41 or more times larger than the normal number of nasal cold actions, and when the cold judgement 4 is determined in step S357, processing of the cold level 4 is performed in step S316e.

As shown in FIG. 36, the second cold judgement on October 31 is 1, the first cold judgement on November 1 is 2, and the second cold judgement on November 1 is 1.

In step S358, the history of the nasal cold action detection counter is updated, and the processing returns to step S312.

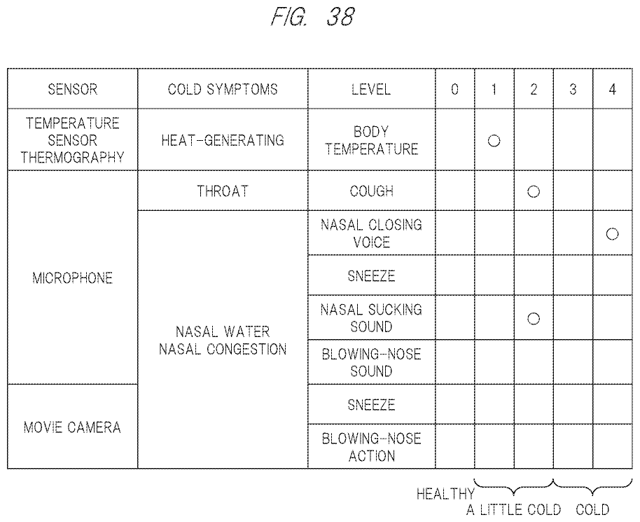

However, since the judgement of the number of times of nasal cold sound or the number of times of nasal cold action may make a cold judgement for symptoms other than cold such as pollinosis or allergic rhinitis, it is desirable to use it in combination with the judgement of febrile cold or the judgement of the number of times of cough. FIG. 38 shows an example of comprehensively determining abstracted data from the cold level derived from each sensing result.

The abstracted data of the cold judgement is comprehensively determined based on a plurality of pieces of information such as a nasal closing cold judgement level, a cough frequency cold judgement level, and a nasal cold sound frequency judgement level. As shown in FIG. 38, the cold level judged by each cold symptom is different, i.e., the cold level 1 in the body temperature cold judgement, the cold level 2 in the cough frequency cold judgement, the cold level 4 in the nasal closing cold judgement, and the cold level 2 in the nasal cold sucking frequency judgement, but when judged from the viewpoint of not overlooking the physical condition of the user, the cold level 4 is selected in the overall judgement. Here, Cold Level 0 is healthy, Cold Level 1 and Cold Level 2 are a little cold, and Cold Level 3 and Cold Level 4 are cold.

The comprehensive judgement is performed by outputting abstracted data regardless of a method of weighting the cold level determined by each cold symptom or taking an average value of the level. By the comprehensive judgement, it is possible to continue the cold judgement while complementing each other by using information from the sensing device 240 even when the bi-function device 130 used in the home 100, for example, a device including a temperature sensor or a thermography, is not present in the hotel 200 of the travel destination.

When the abstracted data of the cold judgement is determined, the actuator device 120 or the bi-function device 130 performs an operation corresponding to the cold judgement abstracted data to the user. For example, the bi-function device 130 can increase the air-conditioning setting temperature and the setting humidity or suggest changing the setting to the user who is catching the cold.

An example of the operations of the actuator device 120 and the bi-function device 130 for each cold judgement level (steps S316a to S316e) will be described below.

In the processing of the cold level 0 in step S316a, for example, the air conditioner 130a operates in accordance with the normal temperature setting, and the humidifier dehumidifier operates in accordance with the normal humidity setting.

In the processing of the cold level 1 in step S16b, for example, the air conditioner 130a is set to a temperature 1° C. higher than the normal temperature setting, and the humidifier dehumidifier is set to a humidity 1% higher than the normal humidity setting. The washstand 130b suggests, for example, “Wash your hands before eating” with voice guidance.

In the processing of the cold level 2 in step S316c, for example, the air conditioner 130a is set to a temperature 2° C. higher than the normal temperature setting, and the humidifier dehumidifier is set to a humidity 2% higher than the normal humidity setting. Refrigerator 120e provides voice guidance, for example, suggesting “What about a warm soup?” The rice cooker 120d provides a voice guidance, and proposes, for example, “What about a well-digested rice gruel?” The washstand 130b suggests, for example, “Wash your hands before eating” with voice guidance. The microwave oven 120c provides a voice guidance, for example, “Can you make a steamed towel in a microwave oven?” is proposed.

In the processing of the cold level 3 in step S316d, for example, the air conditioner 130a is set to a temperature 3° C. higher than the normal temperature setting, and the humidifier dehumidifier is set to a humidity 3% higher than the normal humidity setting. The refrigerator 120e provides a voice guidance, for example, “What about a pot with a lot of vegetables?” is proposed. The rice cooker 120d provides a voice guidance, and proposes, for example, “What about a well-digested rice gruel?” The washstand 130b suggests, for example, “Wash your hands, wash your gargles,” in a voice guidance. The microwave oven 120c provides a voice guidance, for example, “Can you make a steamed towel in a microwave oven?” is proposed.

In the processing of the cold level 4 in step S316e, for example, the air conditioner 130a is set to a temperature 5° C. higher than the normal temperature setting, and the humidifier dehumidifier is set to a humidity 5% higher than the normal humidity setting. The refrigerator 120e provides a voice guidance, for example, “What about a hot pan?” is proposed. The rice cooker 120d provides a voice guidance, and proposes, for example, “How about rice miscellaneous at the end of the pot?” The washstand 130b suggests, for example, “Wash your hands, wash your gargles,” in a voice guidance. The microwave oven 120c provides a voice guidance, and proposes, for example, “Can you make egg sake in a microwave oven?”

An example of the operation of the actuator device 220 and the bi-function device 130 when the external environment you moved to is a different environment from each other will be described below. When the user goes out from a warm region to a cold region (carries the health condition information database 111), additional control corresponding to the region to which the user moves is applied.

In the processing of the cold level 0, for example, the air conditioner 130a operates in accordance with the normal temperature setting, the humidifier dehumidifier operates in accordance with the normal humidity setting, while the air conditioner 230a sets the temperature setting to be 2° C. higher than the air conditioner 130a, and the humidifier dehumidifier of the hotel 200 sets the humidity setting to be 5% higher than the humidity setting of the humidifier dehumidifier at home. The refrigerator 220e sets the internal temperature to a low value.

In the cold level 1 treatment, for example, the air conditioner 130a is set to a temperature 1° C. higher than the normal temperature setting, the humidifier dehumidifier is set to a humidity 1% higher than the normal humidity setting, while the air conditioner 230a sets the temperature setting 2° C. higher than the air conditioner 130a, and the humidifier dehumidifier of the hotel 200 sets the humidity setting 5% higher than the humidity setting of the humidifier dehumidifier at home. The refrigerator 220e sets the internal temperature to a low value.

In the processing of the cold level 2, for example, the air conditioner 130a is set to a temperature 2° C. higher than the normal temperature setting, the humidifier dehumidifier is set to a humidity 2% higher than the normal humidity setting, while the air conditioner 230 a sets the temperature setting 2° C. higher than the air conditioner 130a. and the humidifier dehumidifier of the hotel 200 sets the humidity setting 5% higher than the humidity setting of the humidifier dehumidifier at home. The refrigerator 120e suggests “What is the warm soup?” in the voice guidance, but the refrigerator 220e sets the temperature in the refrigerator to be weak.

In the cold level 3 processing, for example, the air conditioner 130a is set to a temperature 3° C. higher than the normal temperature setting, the humidifier dehumidifier is set to a humidity 3% higher than the normal humidity setting, while the air conditioner 230a sets the temperature setting 2° C. higher than the air conditioner 130a, and the humidifier dehumidifier of the hotel 200 sets the humidity setting 5% higher than the humidity setting of the humidifier dehumidifier at home. The refrigerator 120e suggests “What is a pot with a lot of vegetables?” in the voice guidance, but the refrigerator 220e sets the temperature in the refrigerator to be weak.

In the processing of the cold level 4, for example, the air conditioner 130a is set to a temperature 5° C. higher than the normal temperature setting, the humidifier dehumidifier is set to a humidity 5% higher than the normal humidity setting, while the air conditioner 230a sets the temperature setting 2° C. higher than the air conditioner 130a, and the humidifier dehumidifier of the hotel 200 sets the humidity setting 5% higher than the humidity setting of the humidifier dehumidifier at home. The refrigerator 120e suggests “What about the hot pan?” in the voice guidance, but the refrigerator 220e sets the temperature in the cabinet to a low value.

### Third Embodiment

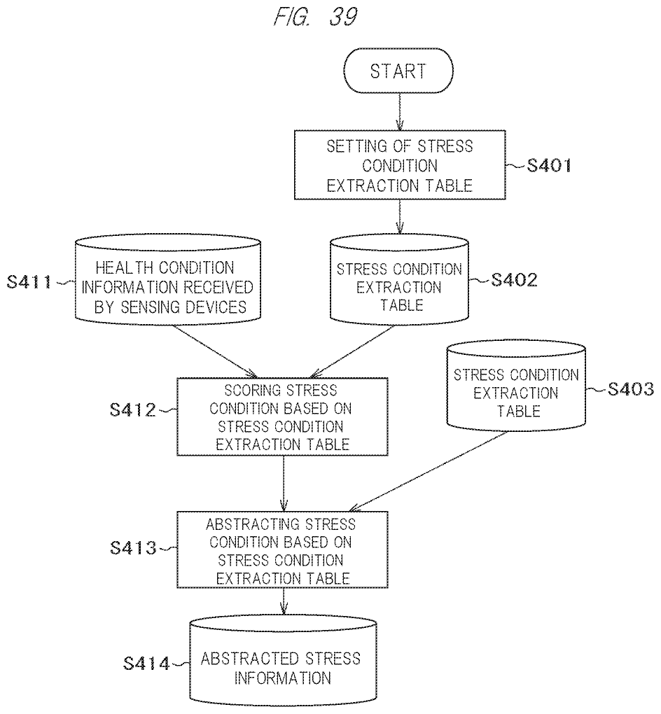

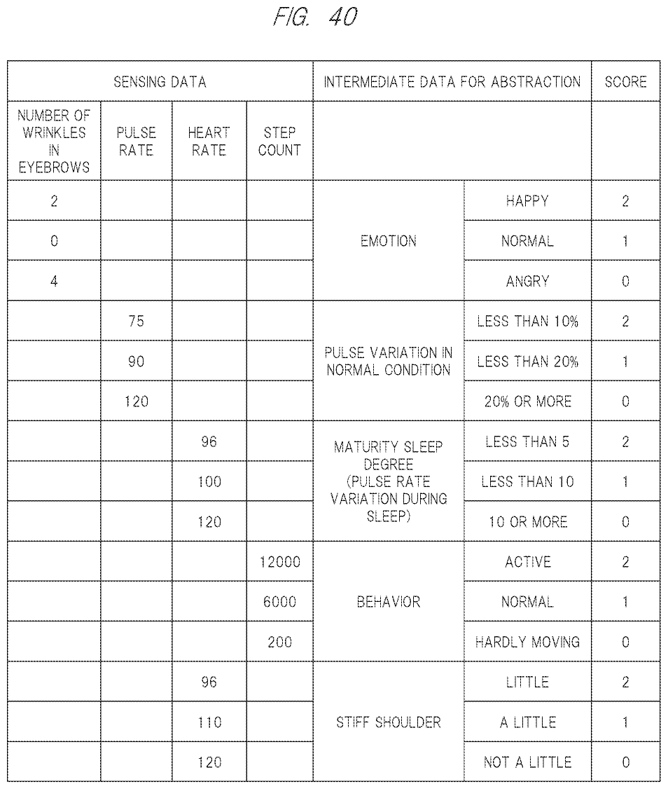

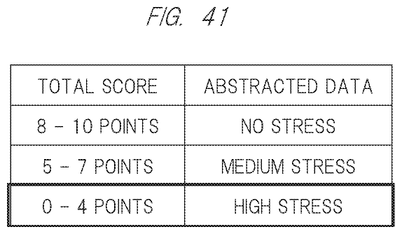

An example of providing health support for stress reduction of a resident will be described with reference to FIGS. 39 to 41. FIG. 39 is a flowchart showing the processing of the stress level abstraction. FIG. 40 is a table for extracting intermediate data of abstraction from sensing data. FIG. 41 is a diagram showing an example of a stress judgement table.

The sensing device 140 and the bi-function device 130 transmit information for determining the stress level of the resident, such as emotion recognition information by voice, emotion recognition information by face recognition of the camera, pulse rate, action amount information, and shoulder stiffness information, to the home server 110.

First, stress information that can be sensed is extracted from the sensing device 140 and the bi-function device 130 existing in the home 100, and sensing data (“number of wrinkles in eyebrows”, “pulse rate”, “heart rate” and “step count”) as shown in FIG. 40 is accumulated (step S411). A table for extracting a stress condition (“emotion,” “pulse rate variation in normal condition,” “mature sleep degree (pulse rate variation in sleep),” “behavior,” and “stiff shoulder”) as shown in “intermediate data for abstraction” of FIG. 40 is set (step S401) and stored (step S401). The “emotion” of the intermediate data for abstraction is classified into “happy”, “normal” and “angry” by “number of wrinkles of eyebrows” or the like, for example. “Normal pulse rate variation” is classified into “less than 10%”, “less than 20%” and “20% or more” by “pulse rate”, for example. The “maturity sleep degree (pulse rate variation during sleep)” is classified into “less than 5”, “less than 10”, and “10 or more” according to, for example, “heart rate”. “Behavior” is classified into “active”, “normal”, and “hardly moving” by, for example, “number of steps”. The “stiff shoulder” is classified into “little”, “a little” and “not a little” by, for example, “heartbeat”.

Next, the home server 110 digitizes (scores) the stress condition of the resident based on the preset stress extraction table as shown in FIG. 40 with respect to the stress information received from the sensing device 140 and the bi-function device 130 (step S412). The stress condition is abstracted based on the stress judgement table (the relationship between the score and the abstracted data) stored in advance as shown in FIG. 41 (step S403) (step S413), and abstracted stress information (abstracted data) is stored (step S414). In the present embodiment, it is abstracted into three stages of high stress, medium stress, and no stress. It is also possible to increase the ultra-high stress level when the high stress condition continues for a long period of time.

The home server 110 then transmits the abstracted data to the actuator device 120 and the bi-function device 130. The actuator device 120 and the bi-function device 130 (e.g., a TV 120b, a CD player, a refrigerator 120e, a washbasin 130b, a lighting 120a, a bath 120f, etc.) each provide health support for the occupant to reduce stress based on the abstracted data received. Examples of the operation of the actuator device 120 and the bi-functional device 130 are described below.

If the abstracted data is “no stress”, the washstand 130d says, for example, “Good! Good for a day!”

For “medium stress”, the TV 120b provides, for example, exercise effective for stress relief, recommended hobbies, stress relief goods. For example, a CD player plays music that is easy to relax during sleep or plays music that images a soft morning when waking up. The refrigerator 120e proposes a menu using, for example, stress-effective foods (foods rich in vitamin B group, vitamin C, etc.). The washstand 130d says, for example, “Oh! you are a little stressed! Don't forget!” The illumination 120a adjusts the brightness step by step during sleep. The bath 120f sets the temperature of the hot water to 40° C. and proposes a bath of at least 10 minutes by voice guidance.

For “high stress,” the TV 120b provides, for example, exercise effective for stress relief, recommended hobbies, stress relief goods information, and institutional information. The CD player plays, for example, music that is easy to relax when sleeping, music that images a soft morning when waking up, or BGM of healing. The refrigerator 120e proposes, for example, a menu using stress-effective foods (foods rich in vitamin B group, vitamin C, and the like), or suggests drinking supplements. The washstand 130d says, for example, “Oh! You have a lot of stress! Don't hold anything alone!” The illumination 120a adjusts the brightness step by step during sleep. The bath 120f sets the temperature of the hot water to 40° C. and proposes a bath of at least 10 minutes by voice guidance.

### Fourth Embodiment

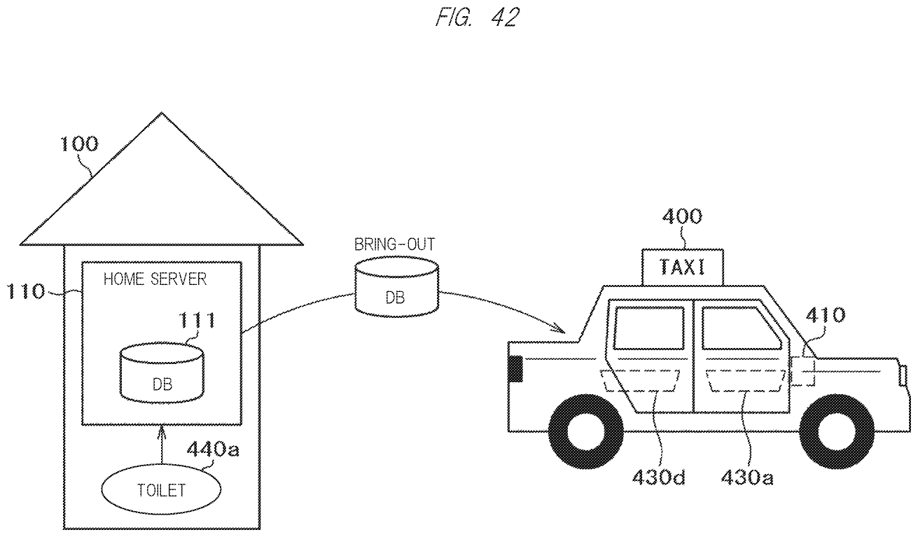

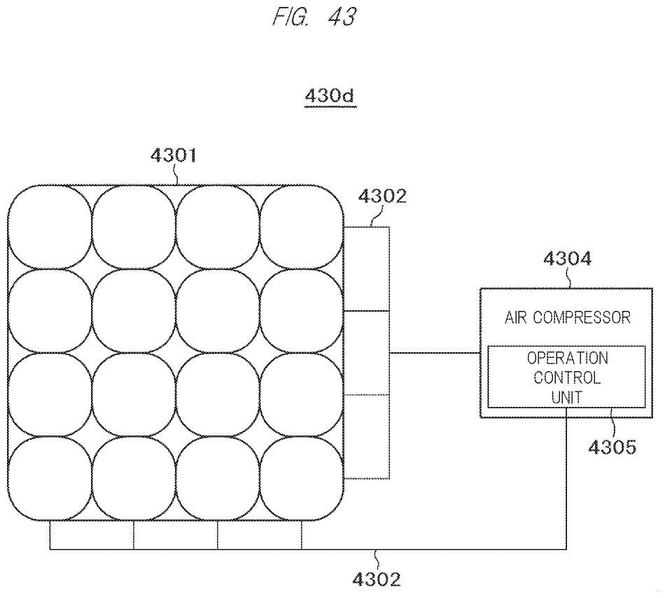

The abstracted data acquired in a daily living space or the like can be applied to an actuator device provided in a non-daily space other than a building. The control of the actuator device provided in the moving means will be described with reference to FIGS. 42 and 43. FIG. 42 is a diagram for explaining an example of bringing out the health condition information database when going out from home using a taxi. FIG. 43 is a diagram showing a configuration of an air cushion which is an example of an actuator device.

The toilet 440a of the home 100 includes a toilet in which a blood sensor is further added to a toilet containing a sensor such as the toilet 140a or the toilet 240a. In the toilet 440a, a function of determining the level of hemorrhoids by a blood sensor is added in addition to a function of determining the level of constipation and diarrhea. The toilet 440a has a function of determining that the user suffers from hemorrhoids when blood components are detected during defecation, determining the level of hemorrhoids based on the blood concentration, and outputting it as abstracted data. The home server 110 stores the level of hemorrhoids as abstracted data in the health condition information database 111. The abstracted data is classified into, for example, “no hemorrhoidal disease”, “hemorrhoidal level 1”, and “hemorrhoidal level 2”. The abstracted data of the passenger is loaded from the health condition information database 111 to the terminal 410 of the taxi 400 when the passenger comes out of the house 100 by using the taxi 400.

The taxi 400 includes an air cushion 430d as shown in FIG. 43. The air cushion 430d includes an air cushion main body 4301 having a plurality of blocks, an air supply tube 4302 for supplying air to each block of the air cushion main body 4301, a block valve control line 4303 for controlling a valve of the air cushion main body 4301, an air compressor 4304 for generating air supplied to each block of the air cushion main body 4301, and an operation control unit 4305 for controlling filling and discharging of air in each block. The air cushion body 4301 has a valve for each of several blocks, and each block can independently control the filling and discharging of air.

When it is determined that the passenger has hemorrhoids, the air cushion 430d discharges only the air in the central portion of the air cushion body 4301 to form a doughnut-shaped cushion. As a result, the passenger can ride on the taxi 400 without worrying about the seating posture or the disease condition and can move comfortably.

An example of the operation control of the in-vehicle device based on the hemorrhoid abstracted data will be described below.

When there is no hemorrhoidal disease, the air conditioner 430a performs normal operation, and the air cushion 430d fills all the blocks with air.

In the case of hemorrhoid level 1, the air conditioner 430a warms the passenger by increasing the blowing ratio of the passenger's foot so that the passenger's foot does not cool. The air cushion 430d exhausts air from the center block and fills the outer peripheral block with air.

In the case of the hemorrhoid level 2, the air conditioner 430a increases the set temperature by 1° C. in addition to the operation of the “hemorrhoid level 1”. The air cushion 430d exhausts air from the center block and fills the outer peripheral block with air. Also, in the case of “cold level 1” or more, the set temperature is raised by 1° C.

Although the invention made by the present inventor has been specifically described based on the embodiments and examples, the present invention is not limited to the embodiments and examples described above, and it is needless to say that the present invention can be variously modified.

What is claimed is:

1. A health support system, comprising:
a sensing device;
an actuator device;
a processing device configured for estimating and obtaining abstracted data of a health condition of a user, based on sensing data sensed by the sensing device, and for controlling the actuator device to improve the health condition of the user, based on the abstracted data.

2. The health support system of claim 1, wherein the sensing device is in a daily living space.

3. The health support system of claim 2, wherein the sensing device classifies, levels and obtains the abstracted data of the health condition of the user based on the sensing data.

4. The health support system of claim 2, wherein a home server classifies and levels and obtains the abstracted data of the health condition of the user based on the sensing data.

5. The health support system of claim 2, wherein the abstracted data is stored in a database, and the database is brought to a non-daily living space different from a daily living space.

6. The health support system of claim 5, wherein the actuator device of the non-daily living space provides health support for the user based on the abstracted data.

7. The health support system according to claim 5, wherein the sensing device of the non-daily living space obtains abstracted data of a health condition of the user based on sensing data sensed by the sensing device of the non-daily living space, the abstracted data of the health condition is added to the database, and the database is brought back to the daily living space.

8. The health support system of claim 5, wherein the actuator device of the non-daily living space reflects an external environment when the actuator device operates.

9. The health support system of claim 2, wherein the sensing device includes an actuator function, or the actuator device includes a sensing function.

10. The health support system of claim 2, wherein the sensing data is acquired by a toilet, and the abstracted data is leveled to normal, first, and second diarrhea levels, or first and second constipation levels of a stomach condition.

11. The health support system of claim 2, wherein the sensing data is a body temperature of the user acquired by a temperature sensor or thermography of the sensing device, and the abstracted data is leveled to normal, first, second, third and fourth cold levels based on the acquired body temperature.

12. The health support system of claim 2, wherein the sensing data is voice information of the user acquired by a microphone of the sensing device, and the abstracted data is leveled to normal, first, second, third, and fourth cold levels based on the acquired voice information.

13. The health support system of claim 12, wherein the voice information is one of a spectrum of a nasal closing voice, a voice waveform pattern of a cough, a voice waveform pattern of a sneeze, a voice waveform pattern of a nasal sucking sound, and a voice waveform pattern of a sneeze.

14. The health support system of claim 2, wherein the sensing data is an action of the user acquired by a movie camera of the sensing device, and the abstracted data is leveled to normal, first, second, third, and fourth cold levels based on the acquired action.

15. The health support system of claim 14, wherein the action is one of a sneezing action and a sneezing action.

16. The health support system of claim 2, wherein the sensing data comprises emotion information and biometric information of the user acquired by the sensing device, and the abstracted data is leveled to normal, first and second stress level based on the acquired emotion information and biometric information.

17. A health support system, comprising:
a first sensing device that is acquiring biometric information of a user;
a first actuator device; and
a first server connected to the first sensing device and the first actuator device via a network, in a daily living space in which the user lives a daily life,
wherein the first sensing device or the first server obtains a first abstracted data based on the biometric information, and controls the first actuator device that is facilitating improvement of the health condition of the user based on the first abstracted data, and
wherein the first abstracted data is classified by estimating a health condition of the user.

18. The health support system of claim 17, further comprising:
a second actuator device; and
a second server or terminal connected to the second actuator device, in a non-daily living space different from the daily living space,
wherein the second server or terminal controls the second actuator device based on the first abstracted data,
wherein the first abstracted data is accumulated in a database of the first server in the daily living space and the database is brought to the non-daily living space.

19. The health support system of claim 18, wherein the second actuator device facilitates improving a health condition of the user based on the first abstracted data stored in the database.

20. The health support system of claim 18, further comprising:
a second sensing device that is acquiring biometric information of the user,
wherein the second sensing device, the second server, or the terminal obtains a second abstracted data of biometric information of the user based on sensing data sensed by the second sensing device, and
wherein the second abstracted data of the biometric information is added to the database, and the database is brought back to the daily living space.
